(19) United States
(12) Patent Application Publication
(10) Pub. No.: US 2006/0194201 A1
(43) Pub. Date: Aug. 31, 2006

(54) Autism gene

(76) Inventors: Jean-Pierre Fryns, Kessel-Lo (BE); Willem Van De Ven, Leuven (BE); Koenraad Devriendt, Holsbeek (BE)

Correspondence Address: CLARK & ELBING LLP, 101 FEDERAL STREET, BOSTON, MA, 02110, US

(21) Appl. No.: 10/530,950

(22) PCT Filed: Oct. 10, 2003
(86) PCT No.: PCT/BE03/00172

§ 371 Date: Apr. 8, 2005

(30) Foreign Application Priority Data
Oct. 10, 2002 (GB) 0223569.5
Nov. 4, 2002 (GB) 0225566.9

(51) Int. Cl. C12Q 1/68

(52) U.S. Cl. 435/6

(57) ABSTRACT

The present invention concerns genes containing mutations associated with autism its onset and development and also to the encoded proteins of said genes associated with autism, its onset and development and the use of said genes, proteins or protein isoforms. The invention thus also relates to methods of screening for, diagnosis and treatment of autism in human subjects e.g., clinical screening, diagnosis, prognosis, therapy and prophylaxis, as will as for drug screening and drug development.

### FIELD OF THE INVENTION

The invention relates to the area of brain anomalies, neural system disorders and particularly to autism.

The present invention concerns genes containing mutations associated with neural system disorders and brain anomalies such as autism its onset and development and also to the encoded proteins of said genes associated with the brain anomalies and autism, its onset and development and the use of said genes, encoded proteins or protein isoforms. The invention thus also relates to methods of screening for, diagnosis and treatment of autism in human subjects e.g., clinical testing or screening, diagnosis, prognosis, therapy and prophylaxis, as well as for drug screening and drug development.

It discloses methods of testing an animal, such as human, thought to have or be predisposed to having neural system disorders or brain anomalies, which comprises detecting the presence of a mutation in the neurobeachin (NBEA) gene and/or its associated promoter. More particularly, the invention relates to detection of the loss and/or alteration of wild-type NBEA genes in cells or tissues and preferably in neural tissues. The method is particularly suitable for testing an animal, such as human, thought to have or be predisposed to autism.

The present invention also relates to the use of a polynucleotide fragment comprising NBEA gene, an allelic variant, minigene or a homologue thereof in the manufacture of a medicament for treating a autism or related neural system disorders. The invention further relates to use of a polypeptide which comprises NBEA or functional homologous thereof in the manufacture of a medicament for treating autism.

### BACKGROUND OF THE INVENTION

Autism is a severe—developmental disorder of the central nervous system characterized by the clinical triad of abnormal language development, disturbance of social skills and particular behavioral features. The disorder starts at young age, has a variable severity and additional medical problems often appear such as mental retardation (75%) or epilepsy (15%). The prevalence of autism is estimated at about 1/1000 to 1/2000. Because of its high prevalence and the need for a lifelong medical and pedagogic supervision, autism is a major burden not only for the families involved but also for public health in general. In 5-10% of the cases, autism is a symptom of a recognizable disorder but in most cases, the cause of autism is not known, and then called 'idiopathic autism”.

Pathogenesis of autism involves a variety of structural brain anomalies have been reported in Magnetic Resonance Imaging (MRI) or postmortem studies, but so far, the most consistent neuropathological findings in autism are abnormalities in the cerebellum, more specifically a decreased number of Purkinje cells were found in 21 of all 23. reported postmortem cases. It is now clear that the cerebellum has an important role in diverse higher cognitive functions, such as the language and emotional control, besides its role in motor control. For these reasons, autism research has recently focused more on the cerebellum. Postmortem studies have implicated the glutamate neurotransmitter system in autism, and reduced levels of the anti-apoptotic protein bcl2 were demonstrated. Nevertheless, a single coherent theory explaining the pathogenesis of autism is lacking.

Family and twin studies have revealed that autism has a genetic origin, inherited as a polygenic disorder, with an estimated 2 to 10 interacting loci. The identification of the genes involved in the origin of autism is an appealing way to gain further insight. At present no genes for idiopathic autism are known. Results of association studies with candidate genes did not yield consistent results. Eight large genome screens failed to define small chromosomal regions harboring susceptibility loci for autism, but several suggestive regions have emerged. Hence, there is a need for new ways to screen for autism. Positional cloning through chromosomal aberrations associated with autism is an alternative means to identify genes involved in autism.

Diagnosis of autism presents difficulties in its own right, and a number of modalities have been proposed primarily based upon psychiatric evaluations. A number of different therapies have been attempted in an effort to cure autism or at least lessen the clinical symptoms thereof. Such have included drug therapies as well as psychiatric care and attempted counseling. In general, results of such treatments have been disappointing, and autism remains very difficult to effectively treat, particularly in severe cases. This invention thus aids in fulfilling these needs in the art.

In a study of 525 patients with idiopathic autism, we now identified four patients with idiopathic autism carrying a de novo balanced chromosomal aberration, three reciprocal translocations, one paracentric inversion. Positional cloning of the breakpoints was initiated; in one patient, (patient CME3) carrying a t(5;13)(q13.3;q14.3), the translocation disrupted the gene coding for neurobeachin (NBEA), located on chromosome 13. Disruption of the gene was shown by means of FISH as well as of Southern blot. The breakpoint on chromosome 5q13.3 did not disrupt any gene.

The de novo occurrence of autism and disruption of the neurobeachin gene are demonstrates that neurobeachin haploinsufficiency is involved in autism.

### SUMMARY OF THE INVENTION

The present invention relates to the use of a polynucleotide fragment encoding NBEA as well as fragments thereof, mutant polynucleotide fragments of the polynucleotide fragment, a recombinant vector comprising such a polynucleotide fragment or mutant polynucleotide fragment, a host cell comprising said polynucleotide fragment or mutant polynucleotide fragment, a host cell comprising a recombinant vector comprising said polynucleotide fragment or mutant polynucleotide fragment, a recombinant or synthetic polypeptide thereto, antibodies specific to said polypeptide, antisense oligonucleotides complementary to said polynucleotide fragment or mutant polynucleotide fragment, interfering RNA (RNAi) interfering with mRNA of said polynucleotide fragment or mutant polynucleotide fragment, pharmaceutical compositions comprising said recombinant or synthetic polypeptide, pharmaceutical compositions comprising said antisense oligonucleotides or said RNAi and pharmaceutical compositions comprising said polynucleotide fragment for use in prophylaxis and/or as a therapeutic agent in animals, particularly humans, as well as uses of said polynucleotide fragment or mutant polynucleotide fragment, antisense oligonucleotides, RNAi, antibodies and/or polypeptides in diagnostic and/or screening assays.

Present invention provides methods for diagnosing and prognosing a neural tissue, cells and preferably neural cells of a human. The invention provides a method of supplying wild-type NBEA gene function to a cell which has lost said gene function. It is yet another object of the invention to provide a kit for determination of the nucleotide sequence of the NBEA gene by the polymerase chain reaction. It is still another object of the invention to provide nucleic acid probes for detection of mutations in the human NBEA gene. It is another object of the invention to provide a method of detecting genetic predisposition to autism. It is still another object of the invention to provide a cDNA molecule encoding the NBEA gene product for use in a kit or for manufacturing a kit to diagnose autism. It is still another object of the invention to provide a cDNA molecule encoding the NBEA gene product or mutated gene product for use in a medicament or to manufacture a medicament to treat austism. It is still another object of the invention to provide a cDNA molecule encoding the NBEA gene product for use in a kit or to manufacture a kit to transfer NBEA gene in cells. It is yet another object of the invention to provide a preparation of the human NBEA protein. These and other objects of the invention are provided by one or more of the embodiments which are described below.

A first aspect of the invention is a method and or a kit of screening for autism in a subject, to establish a diagnosis of autism or give a prognosis. The methods comprise detecting a loss of function, all or part of, of the human NBEA in a tissue and preferably of a nervous tissue of a subject. The loss of function, all or part of, is indicative of the likelihood of occurrence of autism in the subject. Any suitable sample, cell sample or tissue sample of a subject may be used, with nervous samples being more preferred or samples of the central nervous system being most preferred, e.g., cerebellar samples, cerebral samples, and the like.

The detection step may be carried out by determining protein level directly, or by detecting NBEA DNA or RNA changes in expression in a sample obtained from the subject. In addition the detection step can be carried out by assessing the function of NBEA, by measuring either the enzyme activity of the protein or its binding capacities to any substrate, of any kind (organic or inorganic or bio-molecules).

The invention provides a polynucleotide probe suitable for diagnosis of a neural system disorder such as autism in an animal, said polynucleotide probe comprising a polynucleotide sequence which is hybridisable with the NBEA genea and a polynucleotide probe suitable for diagnosis of a neural system disorder such as autism in an animal, said polynucleotide probe comprising a polynucleotide sequence which is hybridisable with a variant NBEA gene, having a deletion, insertion or base substitution which affects transcription and/or translation of the NBEA gene.

As a further aspect, the present invention provides a method of screening for autism in a subject, comprising detecting the presence or absence of a mutation or a polymorphism in the Nbea gene, where the presence of such mutation or polymorphism indicates that the subject is afflicted with, or is at increased risk of developing, autism. Subjects may be heterozygous or homozygous for the mutation. The presence or absence of a mutation or polymorphism may be detected in any suitable cell or tissue sample from the subject, e.g., peripheral white blood cells skin samples, tissue biopsies, and the like.

The polymorphism may be a missense mutation, nonsense mutation, insertion mutation, or deletion mutation and may occur in exon or intron sequences, or in upstream or down-stream regulatory regions of the Nbea gene. Preferably, the mutation results in a functional change of the NBEA protein or in change in expression of the corresponding gene. The mutation screened for is preferably in the mRNA sequence of Nbea. The foregoing method may also be carried out by detecting an ineffective form of the NBEA protein. The detection step for assessing the function or for the presence or absence of a mutation or a polymorphism can be carried out as described above for the neurobeachin protein, mRNA and gene.

The method for screening neural system disorders and more particularly autism in a human comprises: (A) providing chromosomal material from the human; (B) detecting a modification of the NBEA gene or its promotor in the chromosomal material, wherein the modification is selected from a) substitution, b) deletion, c) frame-shift, or d) insertion that causes a loss of biological function in the NBEA gene; and (C) correlating the modification of the gene with a potential for a neural system disorder. The method can also be practiced in anmilas with for instance the mouse NBEA gene or DAKAP550.

The present invention involves thus the isolation of a full length human NBEA cDNA, which will be useful in the production of recombinant NBEA, which can be used for the elucidation of the function of NBEA (e.g. screening for binding partners). Another aspect of the present invention is the use of full length human NBEA cDNA for producing a cellular model to unravel the function of NBEA (in yeast or in mammalian cell-lines such as AtT-20 or Neuro-2A). This model of engineered cell can be used in pharmaceutical screening and for autism and for in vivo modelling of NBEA biochemistry. It can be used as an assay, automated assay or high through put screening assay for identifying agents, compounds or chemical signals that directly or indirectly affect the biochemistry of NBEA, comprising the steps of: growing the cells in appropriate media, said cells comprising an introduced polynucleotide or DNA sequence, an allelic variant, minigene or a homologue thereof, that encodes for NBEA, NBEA isoforms or functional homologues thereof and expresses or overexpresses NBEA or functional homologues thereof, adding the test compound or chemical signal to the media; and measuring the extend to which the NBEA or functional homologues thereof or their function in the cell pathways are affected. NBEA maybe introduced in a cell that has been deleted for endogenous protein kinase (PKA).

The DNA sequence of present invention encoding and capable of expressing a human NBEA or orthologes such as mouse neurobeachin or DAKAP550 a protein of the BEACH-domain containing protein family with a protein kinase A binding domain such as the mammalian LBA or mammalian LvsA will be capable, directly or indirectly, of modulating (e.g. the phosphorylation) of endogenous proteins or introduced proteins, can or may be introduced to establish or bring about a production in cell of the chosen protein kinase A-anchoring protein (AKAP) such as neurobeachin. The invention thus includes also the progeny and all subsequent generations of the cells into which the said DNA sequence(s) were introduced.

This model of engineered cell (eukaryotic cell-lines such as Neuro2A, PC12, AtT-20 and yeast cells) can be used in pharmaceutical screening for agents and for in vivo modelling of mammalian AKAPs and/or BEACH proteins, preferably neurobeachin biochemistry. These cells can also be used for screening compounds that modulate intracellular vesicular transport and of membrane dynamic. It can be used as an assay, automated assay or high through put screening assay for identifying agents, compounds or chemical signals that directly or indirectly affect the biochemistry of neurobeachin and in particular of. its binding capacity to a protein kinase A, type II protein kinase (PKA) or to MARCKS, comprising the steps of: growing the cell line in appropriate media, said cell comprising an introduced polynucleotide or DNA sequence, an allelic variant, minigene or a homologue thereof, that encodes for neurobeachin, neurobeachin isoforms or functional homologues thereof and expresses or overexpresses neurobeachin or functional homologues thereof and wherein said cell comprising a protein that is capable directly or indirectly of being modulated by said neurobeachin and adding the test compound or chemical signal to the media; and measuring the extend to which neurobeachin or functional homologues thereof are affected.

Another aspect of the present invention is thus to provide insight in or a research tool to provide insight in a cellular pathway in which NBEA functions, which, when disrupted or altered, may result in a susceptibility or may cause autism in humans.

The present invention also provides a method and/or kit of screening for autism in a subject, comprising detecting either loss of function (all or part of) of any direct partner of NBEA (PKA, members of the MARCKS protein family, and the like), as well as any protein of the pathway in which NBEA functions (PKA, and the like) or the presence or absence of a mutation or a polymorphism in the corresponding encoding gene.

Since the Drosophila melanogaster DAKAP550 protein (of the BEACH protein family) is closely related to neurobeachin (about 50% similarity), D. melanogaster can be used as an animal model that permits study of the etiology of autism disease and provides a tool to identify new genes involved in the disease pathway, and to identify compounds that may be used to treat or alter the disease progression, lessen its severity or ameliorate symptoms. D. melanogaster can be used to investigate the neurobeachin pathway and the effect of deleted expression, decreased expression or overexpression of the DAKAP550 protein or of introduced neurobeachin to neuronal biochemistry and brain development of the fly. By correlating the phenotype of flies with wild-type DAKAP550 gene or disorders in the DAKAP550 gene, or the phenotype of flies with wild type neurobeachin transgenes and or with disordered NBEA transgenes, can be used to identify other genes involved in this pathway and also means for direct screening for lead candidate compounds for drugs for treatment of autism. Identification of additional genes necessary for neurobeachin or DAKAP550 function can provide additional diagnostic tools for autism. Flies can be mutagenized or treated with a test compound, and those that exhibit a change in phenotype can be identified. If test compounds or mutations responsible for the change in phenotpye (possibly changes in behaviors) are identified, such compounds are candidates for the treatment of autism. Constructs, vectors, plasmids and D. melanogaster strains for comprising the transgene nucleic molecules of neurobeachin are currently available for the man skilled in the art. These D. melanogaster animal models can be used as a screening tool for compounds that normalise or improve a disturbed PKA phosphorylation cascade, intracellular vesicule transport and membrane dynamics and for compounds suitable to treat autism. Also, influencing this pathway through chemical compounds provides a potential treatment strategy for autism.

The present invention also concerns the stereotactic lentiviral vector mediated transfer of transgene polynucleotides encoding a short-hairpin RNA (interfering RNAs or RNAi) specific for NBEA mRNA interfering or close members of the BEACH-domain protein family (BDP) to produce locoregional NBEA or BDP silenced non-human animals to obtain autism models or in non-human animals.

More preferably this method involves stereotactic lentiviral vector mediated transfer of polynucleotides endocing short-hairpin RNA interfering with NBEA mRNA in the brain of various non-human animal species. In its most preferred embodiment the method of this invention involves stereotactic lentiviral vector mediated transfer in the brain of rodents (mice, rat). Such animals with locoregional silenced NBEA may be used as animal models of autism or brain.

Yet another method aspect of the invention is a method and/or kit to create subjects with locoregional RNAi silenced neurobeachin (NBEA) or mutants thereof or to create disease models in non-human animals using stereotactic lentiviral vector mediated transfer of nucleotides which encode short-hairpin RNA that interfere with neurobeachin mRNA in the brain to silence wild type or mutant neurobeachin gene or functional derivatives thereof or other autism associated genes. These animals can be used as animal models of autism.

In present invention in vivo post-transcriptional gene silencing of NBEA is carried out via RNA interference, comprising the use of a lentiviral vector system, wherein said lentiviral vector system encodes RNA. In present invention such post-transcriptional gene silencing via RNA interference is carried out by means of lentiviral vector systems. The lentiviral vector system encodes RNA specific for a certain mRNA of a gene of interest. Said encoded RNA can be partially complementary to a certain mRNA. However, also other suitable transfection systems vectors, retroviral, viral or non viral may be selected for efficient in vivo RNA interference. For instance recently DNA plasmids have been constructed that drive expression of siRNAs from RNA prolymerase III promoters to induce suppression in mammalian cell culture (Paul, C. P., et al. Effective expression of small interfering RNA in human cells. Nat. Biotechnol. 20, 505-508 (2002); Yu, J. Y., et al RNA interference by expression of short-interfering RNAs and hairpin RNAs in mammalian cells. Proc. Natl Acad. Sci. USA 99, 6047-6052 (2002); Paddison, P. J., et al. Short hairpin RNAs (shRNAs) induce sequence-specific silencing in mammalian cells. Genes Dev. 16, 948-958 (2002); Brummelkamp, T. R., et al. A system for stable expression of short interfering RNAs in mammalian cells. Science 296, 550-553 (2002) and Sui, G. et al. A DNA vector-based RNAi technology to suppress gene expression in mammalian cells. Proc. Natl Acad. Sci. USA 99, ₅₅₁₅-₅₅₂₀ (2002)))

Another aspect of the present invention thus comprises a method for NBEA silencing via RNA interference, wherein the lentiviral based vector encodes hairpin RNA specific for a NBEA mRNA of NBEA. In other aspects of the invention the RNA encoded by the lentiviral vector can be single or double stranded.

One aspect of the invention is thus subjects and non-human animals harboring in their neural tissue or brains preferably in the cerebellum a transgene polynucleotide sequence, an allelic variant or a homologue thereof, that encodes for neurobeachin transgene or functional homologues thereof and overexpresses neurobeachin transgene, isoform of neurobeachin or functional homologues thereof locoregional in said neural tissue or said brains for used a therapeutic treatment of autism or as a disease model of autism. Lentiviral vector mediated transfer of neurobeachin gene or functional derivatives thereof may also be used in a transfer to the brain of a subject or as a treatment of autism.

Such transgenes comprising the neurobeachin gene an allelic variant, minigene, a homolog thereof, that encode for neurobeachin, an isoform of neurobeachin or functional homologues thereof or at least a portion thereof are obtainable by a method comprising 1) producing HIV-1-derived vector particles, pseudotyped with the envelope of non related virus, said HIV-1 derived vector particles obtainable by transfecting suitable cells in suitable agents with a suitable packaging plasmid encoding viral gag and pol proteins, a plasmid encoding the envelope of a non related virus and a plasmid encoding neurobeachin gene or an allelic variant, minigene or a homolog thereof which is flanked by LTR's, 2) isolating and concentrating the vector particles 3) redisolving the vector particles in a suitable agent, 4) injecting the vector particle solution in stereotactically defined targets of the brain of a subject.

Pharmaceutical compositions in a pharmaceutically acceptable carrier in the created animal models maybe administered (systemically or locally) and verifying whether the compound alters qualitatively or quantitatively the observed autism pathology or behaviours. Such pharmaceutical compositions should contain a therapeutic amount of at least one compound identified by the method of the present invention. Such compound may be a nucleic acid encoding a protein or fragment of a protein. The pharmaceutically acceptable carrier can be any compatible, non-toxic substance suitable to deliver the compounds to an intended host, animal host or cell line. Sterile water, alcohol, fats, waxes, and inert solids may be used as the carrier. Pharmaceutically acceptable adjuvants, buffering agents, dispersing agents, and the like may also be incorporated into the pharmaceutical compositions. Preparation of pharmaceutical conditions incorporating active agents is well described in the medical and scientific literature (See, for example, Remington's Pharmaceutical Sciences, Mack Publishing Company, Easton, Pa., 16th Ed., 1982, the disclosure of which is incorporated herein by reference). The pharmaceutical compositions just described are suitable for injection in targeted zones and regions of the brain or neural tissue. Thus, the present invention provides compositions for administration to an animal host, where the compositions comprise a pharmaceutically acceptable solution of the identified compound in an acceptable carrier, as described above.

In one embodiment of the present invention a method is provided of diagnosing or prognosing one or more cells, preferably tissue and more preferably neural tissue of a human, comprising:

isolating a cell or tissue from a human;

detecting loss of wild-type NBEA gene coding sequences or their expression products from said cell or tissue, said loss indicating autism.

In another embodiment of the present invention a method is provided for supplying wild-type NBEA function to a cell which has lost said gene function by virtue of mutation in a NBEA gene, comprising:

introducing a wild-type NBEA gene into a cell which has lost said gene function such that said transgene is expressed in the cell.

In another embodiment a method of supplying wild-type NBEA gene function to a cell which has lost said gene function by virtue of a mutation in a NBEA gene, comprising:

introducing a portion of a wild-type NBEA gene into a cell which has lost said gene function such that said portion is expressed in the cell, said portion encoding a part of the NBEA protein which is required for normal function of said cell.

In yet another embodiment a kit is provided for determination of the nucleotide sequence of a NBEA gene by polymerase chain reaction, comprising:

sets of pairs of single stranded DNA primers, said sets allowing synthesis of all nucleotides of the NBEA gene coding said wild-type NBEA gene.

In still another embodiment of the invention a nucleic acid probe is provided which is complementary to human wild-type NBEA gene coding sequences and which can form mismatches with mutant NBEA genes, thereby allowing their detection by enzymatic or chemical cleavage or by shifts in electrophoretic mobility.

In another embodiment a particular nucleic acid probe is provided which hybridizes to a NBEA intron which is subject to insertional mutations in its neural cells.

In yet another embodiment a method is provided of detecting genetic predisposition to autism in a human comprising:

isolating human sample from neural tissue, or any tissue expressing neurobeachin; detecting loss of wild-type NBEA gene coding sequences or their expression products from the sample, said loss indicating predisposition to autism.

In still other embodiments a cDNA molecule comprising a coding sequence of the NBEA gene.

In even another embodiment a preparation of the human NBEA protein is provided which is substantially free of other human proteins for use a medicine, for use to manufacture a medicine or for use in the prophylactic or therapeutic treatment of autism

In yet another embodiment a preparation of the human NBEA protein is provided which is substantially free of other human proteins for use in a diagnostic kit, or for use to manufacture a diagnostic kit for autism.

It is understandable that induced expression expression of siRNAs, e.g. by vector or plasmid transfer can also be used for attenuation of expression of NBEA in vitro in cultured cells, preferably neuronal cell.

The post-transcriptional silencing of NBEA can be obtained in vivo in an organism, such as non-human adult or embryonic animals, preferably mice. Most preferably expression of interfering RNA that attenuate NBEA is induced in the brain in non-dividing cells, such as neurons or neuroendocrine cells. Also post-transcriptional silencing of other NBEA like genes which contain the typical beach domain such as LBA and LvsA and function in vesicular transport and membrane dynamic can be carried according to the method of present invention.

An embodiment, the invention provides a method for neural systems disorders, more particularly autism in an animal, preferably a human, this method comprises: (A) providing biological material from the human; (B) detecting the absence, inappropriate, or modified expression of NBEA gene product using labeled ligands, preferably antibodies to the gene product; and (C) correlating the absence, inappropriate, or modified expression with a potential for neural system defects. The antibodies can be polyclonal or monoclonal.

This invention also provides a recombinant chromosome or engineered polynucleotides comprising a polynucleotide containing a nucleotide sequence, wherein the sequence includes at least one mutation of the NBEA gene, wherein the mutation is selected from a) substitution, b) deletion, c) frame-shift, d) aberrent insertion, e) altered epigenetic control, or f) site-directed mutagenesis that causes a loss of biological function in the NBEA gene.

This invention also provides a cell, preferably a neural cell containing the recombinant chromosome or engineered polynucleotides. The chromosome can be a neural cell chromosome. The neural cell can be derived from an immortal cell line or a stem cell line or a neural stem cell line (including human), and in one embodiment the chromosome can be a stem cell chromosome. The mouse stem cell can be derived, for example, from stem cell line CK35, stem cell line 5b17, or stem cell line 8b21. A β-gal gene can be substituted for the NBEA gene, wherein the stem cell also contains genetic material for selection in selection media.

This invention further provides a method of simultaneously monitoring the expression (e.g. detecting and/or quantifying the expression) of the NBEA gene. The method involves providing a pool of target nucleic acids comprising mRNA transcripts of one or more of these genes, or nucleic acids derived from the mRNA transcripts, hybridizing the pool of nucleic acids to oligonucleotide probes, wherein the oligonucleotide probes are complementary to the mRNA transcripts or nucleic acids derived from the mRNA transcripts, and quantifying the hybridized nucleic acids. The pool of target nucleic acid can be one on which the concentration of the target nucleic acids (mRNA transcripts) or nucleic acids derived from the mRNA transcripts is proportional to the expression levels of the NBEA gene. Microfabricated arrays of large numbers of different oligonucleotide probes (e.g. DNA chips) may effectively be used to detect the presence or absence of the target nucleic acid sequences and to quantify the relative abundance of the target sequences in a complex nucleic acid pool.

Further, this invention provides a chimeric mouse having at least one cell, which is progeny of the stem cells of the invention. The chimeric mouse can be derived from a morula of a CD-1 mouse, such as a morula of a C57BL/6 mouse.

This invention also provides a engineered neural cell comprising a vector comprising the NBEA gene. The NBEA gene can be under control of a neural-specific promoter. The neural cell can be from a wild-type animal. A NBEA gene can be modified, wherein the mutation is selected from a) substitution, b) deletion, c) frame-shift, d) insertion, or e) site-directed mutagenesis that causes a loss of biological function in a NBEA gene. The NBEA gene of the native cell can be altered through a naturally occurring mutation.

Further, this invention provides a method of making a recombinant neural cell comprising: (A) providing a neural cell; (B) modifying a NBEA gene or the promoter of the NBEA gene in the neural cell, wherein the mutation is selected from a) substitution, b) deletion, c) frame-shift, and d) insertion that causes a loss of biological function in the gene; and (C) selecting modified cells.

In addition, this invention provides a method of screening for therapeutic compounds comprising: (A) providing a cell of the invention; (B) introducing to the cell a compound to be screened; and (C) correlating change of the cells with the activity of the compound.

The method of screening for therapeutic compounds is also provided. The method comprises: (A) providing an animal or animal embryo of the invention; (B) introducing to the animal or animal embryo a compound to be screened; and (C) correlating a change in the development of the nervous system with the activity of the compound.

The change in the nervous system can be alteration of number of Purkinje cells of the glutamate neurotransmitter system, of the levels of the anti-apoptotic protein bcl2 of the type II protein kinase A phosphorylation pathway, of the intracellular vesicular transport and/or of membrane traffic.

The invention provides these same embodiments relating to the mouse NBEA gene instead of the human NBEA gene.

### Brief Description of the Drawings

This application includes FIGS. 1-7

FIG. 1. shows a FISH analysis of metaphase chromosomes of VC cells of the autistic patient using cosmid clone 25I17. Hybridization signals are marked by arrows with a chromosome identity label

FIG. 2. shows a map of the breakpoint (BP) region in the autistic patient. (A) FISH analysis. At the BAC level, clone 307O13 is proximal and 66B8 distal to the BP. At the level of cosmids (obtained by cosmid library screening with 66B8_probe2, 6 and 8, cosmids 32G24 and 7H10 are proximal, and 4B16 is distal to the BP. Cosmids 25I17 and 27F13 span the BP. (B) Restriction map used for Southern blot analysis (FIG. 4) with 66B8_South probe; the BP is narrowed down to a 2.8 kb HindIII/BamHI restriction fragment.

FIG. 3. shows a southern blot analysis of genomic DNA from the autistic patient (P) and a control individual (C). Genomic DNA was digested with the mentioned restriction enzymes. Using probe 66B8_South, rearranged fragments are observed for the EcoRI (5.9 kb), HindIII (8.7 kb) and PstI (11.5 kb) digestions in the patient, in addition to the 8.0 kb, 6.4 kb and 14.7 kb wild type fragments, respectively. In the control, only the wild type fragment is visible.

FIG. 4. shows the expression pattern of the hNbea transcript. Northern blots of total RNA from human tissues (Clontech). The probe. used for hybridization is hNbea-ex56. Tissue abbreviations are as followed: B, brain; H, heart; K, kidney; L, liver, Lu, lung; P, pancreas; Pl, placenta; S, skeletal muscle.

FIG. 5. shows the expression of eurobeachin in mouse embryos. Whole mount in situ hybridizations of developing embryos. (A) Ubiquitous expression at stage E7.5. (B) Expression In the limbs, the tail, the branchial arches, and the nose at stage E10.5.

FIG. 6. shows the expression of Neurobeachin in the hippocampus of adult mouse. (A) In situ hybridization. (B) Immunohistochemistry.

FIG. 7. shows the expression of Neurobeachin in the cerebellu m of adult mouse. (A) In situ hybridization. (B) Immunohistochemistry.

### DETAILED DESCRIPTION OF PREFERRED EMBODIMENTS

Generally, the nomenclature used hereafter and the laboratory procedures in cell culture, molecular genetics, and nucleic acid chemistry and hybridisation described below are those well known and commonly employed in the art. All nucleotide sequences shown herein are presented from the 5′ to the 3+ direction. Standard nucleotide abbreviations are used. Except as otherwise indicated, standard techniques may be used for histology, polynucleotide synthesis, cell culture, production and manipulation of cloned genes, vectors, transformed cells (and cell culture) and recombinant DNA technology according to the present invention. Such techniques are known to those skilled in the art (see e.g., SAMBROOK et al., EDS., MOLECULAR CLONING: A LABORATORY MANUAL 2d ed. (Cold Spring Harbor, N.Y. 1989); F. M. AUSUBEL et al, EDS., CURRENT PROTOCOLS IN MOLECULAR BIOLOGY (Green Publishing Associates, Inc. and John Wiley & Sons, Inc., New York).

Generally, enzymatic reactions oligonucleotide synthesis, and purification steps are performed according to the manufacturer's specifications. The techniques and procedures of stereotactic surgery are generally performed according to conventional methods in the art and various general references which are provided throughout this document. The procedures therein are believed to be well known in the art and are provided for the convenience of the reader. All the information contained therein is incorporated herein by reference. Unique technologies are detailed and explained in the examples.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

The term “non-dividing cell” used herein means animal cells such as nerve, liver, muscle and bone marrow stem cells.

The term “Transgene” means any piece of DNA which can be inserted into a cell, and preferably becomes part of the genome of the resulting organism (i.e. either stably integrated or as a stable extrachromosomal element). Such a transgene includes genes which are partly or entirely heterologous (i.e. foreign) as well as genes homologous to endogenous genes of the organism. Including within this definition is a transgene created by providing an RNA sequence which is reverse transcribed into DNA and then incorporated into the genome, or an antisense agent or interfering RNA ore a molecule.

The term “transgenic animal” is used herein to mention non-human animals, having a non-endogenous (i.e. heterologous) nucleic acid sequence present as a extrachromosomal element in stably integrated into its germ line DNA (i.e. in the genomic DNA of most or all of its cells). Heterologous nuleic acid is introduced into the germ line of such transgenic animals by genetic manipulation of, for example, embryous or embryonic stem cells of the host animal. A “transgenic” animal is any animal containing cells that bear genetic information received, directly or indirectly, by deliberate genetic manipulation at the subcellular level

The term “animals with locoregional neural transgenes” is used herein to mean non-human animals which overexpresses a exogenous peptide, polypeptide or protein or at least a portion thereof or an interfering RNA in at least one precisely localised region in the brain or other neural tissue after local delivery, preferably stereotactic vector-mediated transfer, of a “heterologous gene” or “heterologous polynucleotide sequence” encoding a exogenous peptide, polypeptide or protein or at least a portion thereof.

The term “locoregional transgenic brains” is used herein to mean brains of non-human animals which overexpresses a exogenous peptide, polypeptide or protein or at least a portion thereof in at least one precisely localised region in the brain or other neural tissue after local delivery, preferably stereotacted vector-mediated transfer, of a “heterologous gene” or “heterologous polynucleotide sequence” encoding a exogenous peptide, polypeptide or protein or at least a portion thereof or encoding short-hairpin RNA (RNAi).

The term “vector” is used herein to mean that a DNA molecule, derived, e.g., from a plasmid or virus, into which fragments of DNA may be inserted or cloned. A vector will contain one or more unique restriction sites and may be capable of autonomous replication in a defined host or vehicle organism such that the cloned sequence is reproducible.

The term “antisense agent” refers to a molecule which interacts directly with intracellular DNA or RNA to achieve a therapeutic effect. Examples of antisense agents include, without limitation, DNA-binding molecules, triple-helix (or triplex) forming agents, ribozymes, and the like. Antisense agents may be prepared from naturally-occurring nucleotides, or may contain modified bases.

As used herein, a “heterologous gene” or “heterologous polynucleotide sequence” is defined in relation to humans or to the animals with locoregional neural transgenes producing such a gene product in targeted zones or regions of the brain or neural tissue. A heterologous polypeptide, also referred to as a xenogenic polypeptide, is defined as a polypeptide having an amino acid sequence or an encoding DNA sequence corresponding to that of a cognate gene found in an organism not consisting of the animals which harbored the locoregional neural transgenes. Thus, a human or an animal with locoregional expression in brains or neural tissue of a neurobeachin gene can be described as harbouring a heterologous neurobeachin gene. A cognate heterologous gene refers to a corresponding gene from another species; thus human neurobeachin is a cognate heterologous gene for mice. A mutated endogenous gene sequence can be referred to as a heterologous gene.

The term “yeast cell” herein is used to mention single-celled fungi of the phylum Ascomycota that reproduce by fission or budding and are capable of fermenting carbohydrates into alcohol and carbon dioxide. Yeast cells of the species Saccharomyces cerevisae are preferred for manipulation to incorporate DNA sequences in accordance with the present invention. Such cells do not normally express a mammalian neurobeachin but are capable of expressing human neurobeachin by introduction of a DNA sequence encoding neurobeachin under the control of appropriate regulatory DNA sequences. The resulting DNA sequence is considered a “recombinant” DNA sequence or a “transgene”.

The term “engineered yeast” is used herein to mention yeast cells, having a transgene or non-endogenous (i.e. heterologous) nucleic acid sequence present as a extrachromosomal element in stably integrated into its germ line DNA (i.e. in the genomic DNA). Heterologous nucleic acid is introduced into the germ line of such engineered by genetic manipulation

The term “introduced DNA sequence” is used herein to denote a DNA sequence that has been introduced into a cell and which may or may not be incorporated into the genome. The DNA sequence may be a sequence that is not endogenous to the chosen type of cell, that is endogenous but is not normally expressed by that cell or that is endogenous and is normally expressed but of which over-expression is desired. The DNA sequence may be introduced by any suitable transfection technique including electroporation, calcium phosphate precipitation, lipofection or other known to those skilled in the art. The sequence may have been introduced directly into the cell or may have been introduced into an earlier generation of the cell.

As used herein, the term “minigene” refers to a heterologous gene construct wherein one or more nonessential segments of a gene are deleted with respect to the naturally-occurring gene. Typically, deleted segments are intronic sequences of at least about 100 basepairs to several kilobases, and may span up to several tens of kilobases or more. Isolation and manipulation of large (i.e., greater than about 50 kilobases) targeting constructs is frequently difficult and may reduce the efficiency of transferring the targeting construct into a host cell. Thus, it is frequently desirable to reduce the size of a targeting construct by deleting one or more nonessential portions of the gene. Typically, intronic sequences that do not encompass essential regulatory elements may be deleted. Frequently, if convenient restriction sites bound a nonessential intronic sequence of a cloned gene sequence, a deletion of the intronic sequence may be produced by: (1) digesting the cloned DNA with the appropriate restriction enzymes, (2) separating the restriction fragments (e.g., by electrophoresis), (3) isolating the restriction fragments encompassing the essential exons and regulatory elements, and (4) ligating the isolated restriction fragments to form a minigene wherein the exons are in the same linear order as is present in the germline copy of the naturally-occurring gene. Alternate methods for producing a minigene will be apparent to those of skill in the art (e.g., ligation of partial genomic clones, which encompass essential exons but which lack portions of intronic sequence). Most typically, the gene segments comprising a minigene will be arranged in the same linear order as is present in the germline gene, however, this will not always be the case. Some desired regulatory elements (e.g., enhancers, silencers) may be relatively position-insensitive, so that the regulatory element will function correctly even if positioned differently in a minigene than in the corresponding germline gene. For example, an enhancer may be located at a different distance from a promoter, in a different orientation, and/or in a different linear order. For example, an enhancer that is located 3′ to a promoter in germline configuration might be located 5′ to the promoter in a minigene. Similarly, some genes may have exons, which are alternatively spliced, at the RNA level, and thus a minigene may have fewer exons and/or exons in a different linear order than the corresponding germline gene and still encode a functional gene product. A cDNA encoding a gene product may also be used to construct a minigene. However, since it is often desirable that the heterologous minigene be expressed similarly to the cognate naturally-occurring non-human gene, transcription of a cDNA minigene typically is driven by a linked gene promoter and enhancer from the naturally-occurring gene. Frequently, such minigene may comprise a transcriptional regulatory sequence (e.g., promoter and/or enhancer) that confers neuron-specific or CNS-specific transcription of the neurobeachin encoding sequences.

The term “agent” is used herein to denote a chemical compound, a mixture of chemical compounds, a biological macromolecule, or an extract made from biological materials such as bacteria, plants, fungi, or animal (particularly mammalian) cells or tissues.

The term “autism” as used herein has its conventional meaning in the art (see, e.g., U.S. Pat. Nos. 5,686,311 and 5,405,943) (applicants specifically intend that all U.S. Patent references cited herein be incorporated herein by reference). In general, autism is a pervasive developmental disorder involving language delay and dysfunction in reciprocal social interaction, and includes a spectrum of disorders that may or may not involve mental deficit or retardation. Thus, high functioning individuals (i.e., individuals with normal intelligence) may be afflicted with autism. Autism is typically considered a life-long disorder, and thus may be present in infant, juvenile, adolescent and adult subjects. Mammalian subjects are preferred, with human subjects being more preferred. The subjects may be male or female, but are preferably male subjects, more preferably, human male subjects.

‘Polynucleotide fragment” as used herein refers to a polymeric form of nucleotides of any length, both to ribonucleic acid sequences and to deoxyribonucleic acid sequences, In principle, this term refers to the primary structure of the molecule, thus this term includes double stranded and single stranded DNA, as well as double and single stranded RNA, and modifications thereof,

In general, the term “a polypeptide” refers to a molecular chain of amino acids with a biological activity, It does not refer to a specific length of the product, and if required it can be modified in vivo and/or in vitro, for example by glycosylation, myristoylation, amidation, carboxylation or phosphorylation; thus inter alia peptides, oligopeptides and proteins are included. The polypeptides disclosed herein may be obtained, for example, by synthetic or recombinant techniques known in the art, Thus the term extends to cover, for example, polypeptides obtainable from various transcripts and splice variants of these transcripts from the NBEA gene.

Additionally, functional domains may be observed in the protein and isolated polypeptides relating to these functional domains may be of particular use. For example, BEACH domain and a Pleckstrine domain have been observed in NBEA. The present invention also relates to polynucleotide fragments comprising a nucleotide sequence encoding such functional domain polypeptides.

It will be understood that for the NBEA nucleotide and polypeptide sequences referred to herein, natural variations can exist between individuals. These variations may be demonstrated by amino acid differences in the overall sequence or by deletions, substitutions, insertions or inversions of amino acids in said sequence. All such variations are included in the scope of the present invention.

As is well known in the art, the degeneracy of the genetic code permits substitution of bases in a codon resulting in a different codon encoding the same amino acid, Consequently, it is clear that any such derivative nucleotide sequence based on the sequences disclosed herein is also included in the scope of the present invention.

The implications for this invention are widespread. A cDNA encoding mutated NBEA protein has been isolated. This discovery of the cDNA encoding mutated NBEA protein enables construction of expression vectors comprising nucleic acid sequences encoding mutated NBEA proteins, and the corresponding RNAs; host cells transfected or transformed with the expression vectors; biologically inactive NBEA proteins as isolated or purified proteins; and antibodies immunoreactive with mutated NBEA proteins. In addition, understanding of the mechanism by which mutated NBEA protein function enables the design of assays to detect substances that affect NBEA protein activity. It will of course be understood that while the various embodiments of this invention may be described with reference to mouse NBEA, the invention applies to the same extent to human NBEA

Thus, as used herein, the term “mutated NBEA proteins” refers to a genus of mutated proteins that exhibit a loss of biological function, and that further encompasses mutated proteins described herein, as well as those mutated proteins having a high degree of similarity (at least 90% homology) with such amino acid sequences, and which proteins lack at least one of the biological activities of naturally occurring NBEA proteins.

The term “purified” as used herein, means that the mutated NBEA proteins and polynucleotides are essentially free of association with other proteins or polypeptides and polynucleotides, for example, as a purification product of recombinant host cell culture or as a purified product from a non-recombinant source.

The term “substantially purified” as used herein, refers to a mixture that contains mutated NBEA proteins and polynucleotides and is essentially free of association with other proteins or polypeptides and polynucleotides, but for the presence of known proteins that can be removed using a specific antibody, and which substantially purified mutated NBEA proteins can be used as antigens.

Variants can comprise conservatively substituted sequences, meaning that a given amino acid residue is replaced by a residue having similar physiochemical characteristics. Examples of conservative substitutions include substitution of one aliphatic residue for another, such as lie, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring mutated NBEA proteins variants are also encompassed by the invention. Examples of such variants are proteins that result from proteolytic cleavage of the mutated NBEA proteins. Variations attributable to proteolysis include, for example, differences in the termini upon expression in different types of host cells, due to proteolytic removal of one or more terminal amino acids from the mutated NBEA proteins. Variations attributable to frame shifting include, for example, differences in the termini upon expression in different types of host cells due to different amino acids. Variation can also result from a chemical mutation by phosphorylation.

As stated above, the invention provides isolated and purified, or homogeneous, mutated NBEA proteins, both recombinant and non-recombinant. Variants and derivatives of native mutated NBEA proteins that can be used as antigens can be obtained by mutations of nucleotide sequences coding for native mutated NBEA proteins.

Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene, wherein predetermined codons can be altered by substitution, deletion, or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

The present invention also includes nucleotide sequences similar to the polynucleotide sequences disclosed herein. It is understood that similar sequences include sequences which remain hybridised to the polynucleotide sequences of the present invention under stringent conditions. Typically, a similar test sequence and a polynucleotide sequence of the present invention are allowed to hybridise for a specified period of time generally at a temperature of between 50 and 70° C. in double strength SSC (2×NaCl 17.5g/I and sodium citrate (SC) at 8.8 g/1) buffered saline containing 0.1% sodium dodecyl sulphate (SDS) followed by rinsing of the support at the same temperature but with a buffer having a reduced SSC concentration, Depending upon the degree of similarity of the sequences, such reduced concentration buffers are typically single strength SSC containing 0.1% SDS, half strength SSC containing 0.1% SDS and one tenth strength SSC containing 0.1% SDS, Sequences having the greatest degree of similarity are those the hybridisation of which is least affected by washing in buffers of reduced concentration. It is most preferred that the similar and inventive sequences are so similar that the hybridisation between them is substantially unaffected by washing or incubation in one tenth strength SSC containing 0.1% SDS.

Furthermore, fragments derived from the NBEA gene which still display NBEA specific properties or NBEA specific properties are, also included in the present invention.

“NBEA specific properties’ is understood to relate to biological functions which are attributable to naturally-occurring NBEA gene and “NBEA specific properties’ is understood to relate to biological functions which are attributable to naturally-occurring NBEA protein. This may include fusion proteins.

All such modifications mentioned above resulting in such derivatives of NBEA are covered by the present invention so long as the characteristic NBEA properties remain substantially unaffected in essence.

The mouse neurobeachin cDNA (mNbea cDNA) and the corresponding protein (mNBEA) are known (Wang, X., et al. J. Neurosci. 20 (23), 8551-8565 (2000)) DBSOURCE Accession Number Y18276.1; see also “The neurobeachin gene (Nbea) identifies a new region of homology between mouse central chromosome 3 and human chromosome 13q13” (Gilbert, D. J. Mamm. Genome 10 (10), 1030-1031 (1999)). The full-length cDNA sequence of the human orthologue of neurobeachin is known DBSOURCE Accession Number AF467288 or NM_{—}015678.

Oligonucleotide probes that specifically bind to an NBEA DNA or RNA that contains a mutation or a polymorphism, but do not bind to a NBEA DNA or RNA that does not contain the mutation or polymorphism, may be produced in accordance with known techniques. Such probes are typically from 5 or 10-nucleotides in length to 20, 30 or 50 nucleotides in length or more. Such probes may be natural or synthetic.

As noted above, the present invention provides a method of screening (e.g., diagnosing or prognosing) for autism in a subject (typically, a human subject). The method comprises detecting the presence or absence of a mutation or a polymorphism in the subject. The presence of such a mutation indicates that the subject is afflicted with autism or is at risk of developing autism. Suitable subjects include those which have not previously been diagnosed as afflicted with autism, those which have previously been determined to be at risk of developing autism, and those who have been initially diagnosed as being afflicted with autism where confirming information is desired. Thus, subjects may be of any age, including adult, adolescent, juvenile, infant, and even prenatal or in utero subjects. Preferably, the subjects are male subjects.

Affliction with autism is more likely if a mutation described above is present. A subject with the mutation has increased risk of developing autism over subjects in which the mutation is absent. A subject who is “at increased risk of developing autism” is one who is predisposed to the disease, has genetic susceptibility for the disease or is more likely to develop the disease than subjects in which the mutation is absent.

Further, the methods of the present invention can be used to aid in determining the prognosis of a subject afflicted with or at risk for autism based on the observation of how many alleles containing the mutation are detected in the subject. The subject's prognosis is more negative if the presence of the mutation is detected than if it is absent. In particular embodiments, the subject's prognosis is most negative if the presence of more than one allele containing the mutation is detected (i. e., if the subject is homozygous as opposed to heterozygous). In other embodiments, homozygous subjects do not appear to be at a substantially higher risk than heterozygous subjects.

It is contemplated that the methods described herein be used in conjunction with other clinical diagnostic information known or described in the art that are used in the evaluation of subjects with autism or suspected to be at risk for developing such disease.

The step of detecting a mutation or a polymorphism may be carried out either directly or indirectly by any suitable means. A variety of techniques are known to those skilled in the art. All generally involve the step of collecting a sample of biological material containing DNA, and then detecting whether or not the subject possesses DNA containing such a mutation from that sample.

Any biological sample that contains the nucleic acid (e.g., DNA, RNA) of that subject may be employed, including tissue samples and blood samples, with blood cells being a particularly convenient source.

Determining the presence or absence of nucleic acid containing a mutation or a polymorphism may be carried out with an oligonucleotide probe labeled with a suitable detectable group, or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for a mutation or a polymorphism.

Amplification of a selected, or target, nucleic acid sequence may be carried out by any suitable means. See generally D. Kwoh and T. Kwoh, Am. Biotechnol. Lab. 8, 14-25 (1990). Examples of suitable amplification techniques include, but are not limited to, polymerase chain reaction (see generally G. Walker et al., Proc. Natl. Acad. Sci. U.S.A. 89,392-396 (1992); G. Walker et al., Nucleic Acids Res. 20, 169 1-1696 (1992)), transcription-based amplification (see D. Kwoh et al., Proc. Natl. Acad Sci. U.S.A. 86, 1173-1 177 (1989)). Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present.

Kits for determining if a subject is or was afflicted with or is or was at increased risk of developing autism will include at least one reagent specific for detecting for the presence or absence of a mutation or polymorphism, and instructions for observing that the subject is or was afflicted with or is or was at increased risk of developing autism if the presence of the mutation is detected. The kit may optionally include a nucleic acid or oligonucleotide probe for detection of the mutation in a manner such as described above. The test kit may be packaged in any suitable manner, typically with all elements in a single container or package along with a sheet of printed instructions for carrying out the test.

An isolated DNA as described above may be provided in a suitable vector, including but not limited to plasmids, viral vectors, yeast artificial chromosomes, bacterial artificial chromosomes, naked DNA vectors, and the like. Preferably, the vector is a plasmid. The present invention also provides cells that have been transformed with the vector, and preferably express the DNA therein. Cells according to the present invention may be any suitable cells for replicating and expressing the DNA, including but not limited to bacterial cells, yeast cells, plants cells, and animal cells (e.g., avian, insect and mammalian cells). Mammalian (e.g., human, mouse, rat, canine, simian), insect and bacterial cells are preferred. Such cells may be grown in cell culture using standard techniques. The cells of the invention may be used to screen new oligonucleotide probes for use in the diagnostic and prognostic techniques described above. In addition, such cells may be used to screen for compounds that affect the mutations described herein, which compounds are then candidate compounds for treating autism.

According to the diagnostic and prognostic method of the present invention, loss of the wild-type gene is detected. The loss may be due to either insertional, deletional or point mutational events. The finding of NBEA mutations thus provides both diagnostic and prognostic information.

A NBEA allele which is not deleted (e.g., that on the sister chromosome to a chromosome carrying a NBEA deletion) can be screened for other mutations, such as insertions, small deletions, and point mutations.

Detection of point mutations may be accomplished by molecular cloning of the allele (or alleles) present in the neural tissue of test subject and sequencing that allele(s) using techniques well known in the art. Alternatively, the polymerase chain reaction can be used to amplify gene sequences directly from a genomic DNA preparation from neural tissue. The DNA sequence of the amplified sequences can then be determined. The polymerase chain reaction itself is well known in the art. See, e.g., Saiki et al., Science, Vol. 239, p. 487, 1988; U.S. Pat. No. 4,683,203; and U.S. Pat. No. 4,683,195. Specific primers which can be used in order to amplify the gene will be discussed in more detail below. Insertions and deletions of genes can also be detected by these techniques. In addition, restriction fragment length polymorphism (RFLP) probes for the gene or surrounding marker genes can be used to score loss of an allele or an insertion in a polymorphic fragment. Other techniques for detecting insertions and deletions as are known in the art can be used.

Loss of wild-type genes can also be detected on the basis of the loss of a wild-type expression product of the gene. Such expression products include both the mRNA as well as the protein product itself. Point mutations may be detected by amplifying and sequencing the mRNA or via molecular cloning of cDNA made from the mRNA. The sequence of the cloned cDNA can be determined using DNA sequencing techniques which are well known in the art.

Mismatches, according to the present invention are hybridized nucleic acid duplexes which are not 100% homologous. The lack of total homology may be due to deletions, insertions, substitutions or frameshift mutations. Mismatch detection can be used to detect point mutations in the gene or its mRNA product. An example of a mismatch cleavage technique is the RNase protection method, which is described in detail in Winter et al., Proc. Natl. Acad. Sci. USA, Vol. 82, p. 7575, 1985 and Meyers et al., Science, Vol. 230, p. 1242, 1985. In the practice of the present invention the method involves the use of a labeled riboprobe which is complementary to the human wild-type gene coding sequence. The riboprobe and either mRNA or DNA isolated from the neural tissue are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the NBEA mRNA or gene but can be a segment of either. If the riboprobe comprises only a segment of the NBEA. mRNA or gene it will be desirable to use a number of these probes to screen the whole mRNA sequence for mismatches.

In similar fashion, DNA probes can be used to detect mismatches, through enzymatic or chemical cleavage. See, e.g., Cotton et al., Proc. Natl. Acad. Sci. USA, vol. 85, 4397, 1988; and Shenk et al., Proc. Natl. Acad. Sci. USA, vol. 72, p. 989, 1975. Alternatively, mismatches can be detected by shifts in the electrophoretic mobility of mismatched duplexes relative to matched duplexes. See, e.g., Cariello, Human Genetics, vol. 42, p. 726, 1988. With either riboprobes or DNA probes, the cellular mRNA or DNA which might contain a mutation can be amplified using PCR (see below) before hybridization. Changes in DNA of the NBEA gene can also be detected using Southern hybridization, especially if the changes are gross rearrangements, such as deletions and insertions.

DNA sequences of the NBEA gene from neural tissue which have been amplified by use of polymerase chain reaction may also be screened using allele-specific probes. These probes are nucleic acid oligomers, each of which contains a region of the NBEA gene sequence harboring a known mutation. For example, one oligomer may be about 30 nucleotides in length, corresponding to a portion of the NBEA gene sequence. By use of a battery of such allele-specific probes, the PCR amplification products can be screened to identify the presence of a previously identified mutation in the NBEA gene. Hybridization of allele-specific probes with amplified NBEA sequences can be performed, for example, on a nylon filter. Hybridization to a particular probe under stringent hybridization conditions indicates the presence of the same mutation in the neural tissue as in the allele-specific probe.

Loss of NBEA mRNA expression can be detected by any technique known in the art. These include Northern analysis, PCR amplification and RNase protection. Diminished mRNA expression indicates a loss of the wild-type NBEA gene.

Loss of wild-type NBEA genes can also be detected by screening for loss of wild-type NBEA protein. For example, monoclonal antibodies immunoreactive with NBEA can be used to screen a tissue. Lack of antigen would indicate a NBEA mutation. Antibodies specific for mutant alleles could also be used to detect mutant NBEA gene product. Such immunological assays could be done in any convenient format known in the art. These include Western blots, immunohistochemical assays and ELISA assays. Any means for detecting an altered NBEA protein can be used to detect loss of wild-type NBEA genes. Finding a mutant NBEA gene product indicates loss of a wild-type NBEA gene.

Mutant NBEA genes or gene products can possibly be detected in other human body samples than neural tissue. The same techniques discussed above for detection of mutant NBEA genes or gene products in neural tissues can be applied to other body samples. In addition, the NBEA gene product itself may be secreted into the extracellular space and found in these body samples. By screening such body samples, a simple early diagnosis can be achieved for autism detection.

The diagnostic method of the present invention is useful for clinicians so that they can decide upon an appropriate course of treatment.

The primer kit of the present invention is useful for determination of the nucleotide sequence of the NBEA gene using the polymerase chain reaction. The kit comprises a set of pairs of single stranded DNA primers which can be annealed to sequences within or surrounding the NBEA gene in order to prime amplifying DNA synthesis of the NBEA gene itself. The complete set allows synthesis of all of the nucleotides of the NBEA gene coding sequences, i.e., the exons. The set of primers can also allow allow synthesis of both intron and exon sequences, to include NBEA mutations in the NBEA introns. The kit can also contain DNA polymerase, preferably Taq polymerase, and suitable reaction buffers. Such components are known in the art.

In order to facilitate subsequent cloning of amplified sequences, primers may have restriction enzyme sites appended to their 5′ ends. Thus, all nucleotides of the primers are derived from NBEA sequences or sequences adjacent to NBEA except the few nucleotides necessary to form a restriction enzyme site. Such enzymes and sites are well known in the art. The primers themselves can be synthesized using techniques which are well known in the art. Generally, the primers can be made using synthesizing machines which are commercially available. Given the sequence of the NBEA open reading frame, design of particular primers is well? within the skill of the art.

The nucleic acid probes provided by the present invention are useful for a number of purposes. They can be used in Southern hybridization to genomic DNA and in the RNase protection method for detecting point mutations already discussed above. The probes can be used to detect PCR amplification products. They may also be used to detect mismatches with the NBEA gene or mRNA using other techniques. Mis matches can be detected using either enzymes (e.g., S1 nuclease), chemicals (e.g., hydroxylamine or osmium tetroxide and piperidine), or changes in electrophoretic mobility of mismatched hybrids as compared to totally matched hybrids. These techniques are known in the art. See, Cotton, supra, Shenk, supra, Myers, supra, Winter, supra, and Novack et al., Proc. Natl. Acad. Sci. USA, vol. 83, p. 586, 1986. Generally, the probes are complementary to NBEA gene coding sequences, although probes to certain introns are also contemplated. An entire battery of nucleic acid probes can be used to compose a kit for detecting loss of wild-type NBEA genes, the kit allowing for hybridization to the entire NBEA gene. The probes may overlap with each other or be contiguous.

If a riboprobe is used to detect mismatches with mRNA, it is complementary to the mRNA of the human wild-type NBEA gene. The riboprobe thus is an anti-sense probe in that it does not code for the NBEA protein because it is of the opposite polarity to the sense strand. The riboprobe generally will be radioactively labeled which can be accomplished by any means known in the art. If the riboprobe is used to detect mismatches with DNA it can be of either polarity, sense or anti-sense. Similarly, DNA probes also may be used to detect mismatches. Nucleic acid probes may also be complementary to mutant alleles of NBEA gene. These are useful to detect similar mutations in other patients on the basis of hybridization rather than mismatches. These are discussed above and referred to as allele-specific probes. As mentioned above, the NBEA probes can also be used in Southern hybridizations to genomic DNA to detect gross chromosomal changes such as deletions and insertions. The probes can also be used to select cDNA clones of NBEA genes from neural tissues from autistic subjects and from normal neural tissues. In addition, the probes can be used to detect NBEA mRNA in tissues to determine if expression is diminished as a result of loss of wild-type NBEA genes. Provided with the NBEA coding sequence, design of particular probes is well within the skill of the ordinary artisan.

According to the present invention a method is also provided of supplying wild-type NBEA function to a cell which carries mutant NBEA alleles. The wild-type NBEA gene or a part of the gene may be introduced into the cell in a vector such that the gene remains extrachromosomal. In such a situation the gene will be expressed by the cell from the extrachromosomal location. If a gene portion is introduced and expressed in a cell carrying a mutant NBEA allele, the gene portion should encode a part of the NBEA protein which is required for normal functioning of the cell. More preferred is the situation where the wild-type NBEA gene or a part of it is introduced into the mutant cell in such a way that it recombines with the endogenous mutant NBEA gene present in the cell. Such recombination requires a double recombination event which results in the correction of the NBEA gene mutation. Vectors for introduction of genes both for recombination and for extrachromosomal maintenance are known in the art and any suitable vector may be used. Methods for introducing DNA into cells such as electroporation, calcium phosphate co-precipitation and viral transduction are known in the art and the choice of method is within the competence of the routineer. Cells transformed with the wild-type NBEA-gene can be used as model systems to study autism related to the cellular dysfunction and drug treatments which promote the reinstatement of normal cellular functions.

Polypeptides which have NBEA function can be supplied to cells which carry mutant or missing NBEA alleles. The NBEA protein can be produced by expression of the cDNA sequence in bacteria or eukaryotic cells (e.g. yeast, CHO cells), for example, using known expression vectors. Alternatively, NBEA can be extracted from NBEA-producing mammalian cells such as brain cells or cell-lines (AtT-20 or Neuro-2A). The preparation is substantially free of other human proteins. This is most readily accomplished by synthesis in a microorganism or in vitro. Active NBEA molecules can be introduced into cells by microinjection or by use of liposomes, for example. Alternatively, some such active molecules may be taken up by cells, actively or by diffusion. The application of NBEA gene product may be sufficient to restore normal cell function. Other molecules with NBEA activity may also be used to effect.

Predisposition to autism can be ascertained by testing neural tissues of humans for mutations of NBEA gene. For example, a person who has inherited a germline NBEA mutation would be prone to develop autism. This can be determined by testing DNA from tissue, preferably neural tissue of the person's body and most preferably from the central nervous system. In addition, prenatal diagnosis can be accomplished by testing fetal cells or amniotic fluid for mutations of the NBEA gene. Loss of a wild-type NBEA allele, whether for example, by point mutation or by deletion, can be detected by any of the means discussed above.

Molecules of cDNA according to the present invention are NBEA gene coding molecules, that can be made by reverse transcriptase using the NBEA mRNA as a template. These molecules can be propagated in vectors and cell lines as is known in the art. The cDNA can also be made using the techniques of synthetic chemistry given the sequence disclosed herein.

Typical techniques for detecting the mutation may include restriction fragment length polymorphism, hybridisation techniques, DNA sequencing, exonuclease resistance, micro sequencing, solid phase extension using ddNTPs, extension in solution using ddNTPs, oligonucleotide ligation assays, methods for detecting single nucleotide polymorphisms such as dynamic allele-specific hybridisation, ligation chain reaction, mini-sequencing, DNA “chips”, allele-specific oligonucleotide hybridisation with single or dual-labelled probes merged with PCR or with molecular beacons, and others.

The information presented herein may also be used to genetically manipulate the wild-type NBEA gene, mutant NBEA gene or derivatives thereof, for example to clone the gene by recombinant DNA techniques generally known in the art. Cloning of homologous genes from other species of mammal may be performed with this information by widely known techniques; for example, suitable primers may be designed to a consensus region and/or functional domains of the sequence shown in the examples of this application and such primers used as probes for cloning homologous genes from other organisms.

Moreover, mammalian NBEA mutant and wild-type nucleotide sequences of the present invention are preferably linked to expression control sequences. Such control sequences may comprise promoters, operators, inducers, ribosome binding sites etc, Suitable control sequences for a given host may be selected by those of ordinary skill in the art.

A nucleotide sequence according to the present invention can be ligated to various expression-controlling DNA sequences, resulting in a so-called recombinant nucleic acid molecule. Thus, the present invention also includes an expression vector comprising an expressible NBEA mutant or wild-type nucleotide sequence. Said recombinant nucleic acid molecule can then be used for transformation of a suitable host. Such recombinant nucleic acid molecules are preferably derived from for example, plasmids, or from nucleic acid sequences present in bacteriophages or viruses and are termed vector molecules.

Specific vectors which can be used to clone nucleotide sequences according to the invention are known in the art (eg, Rodriguez and Denhardt, editors, Vectors: A survey of molecular cloning vectors and their uses, Butterworths, 1988).

The methods to be used for the construction of a recombinant nucleic acid molecule according to the invention are known to those of ordinary skill in the art and are inter alia set forth in Sambrook et al, Molecular Cloning: A Laboratory Manual, Cold Spring Harbour Laboratory, 1989

The present invention also relates to a transformed cell comprising the mutant or wild-type nucleic acid molecule in an expressible form, “Transformation”, as used herein, refers to the introduction of a heterologous nucleic acid sequence into a host cell in vivo, ex vivo or in vitro irrespective of the method used, for example, by calcium phosphate co-precipitation, direct uptake or transduction. The heterologous nucleic acid sequence may be maintained through autonomous replication or alternatively may be integrated into the host's genome. The recombinant DNA molecules are preferably provided with appropriate control sequences compatible with the designated host which can regulate the expression of the inserted nucleic acid sequence.

The most widely used hosts for expression of recombinant nucleic acid molecules are bacteria, yeast, insect cells and mammalian cells. Each system has advantages and disadvantages in terms of the vector used, potential ease of production and purification of a recombinant polypeptide and authenticity of product in terms of tertiary structure, glycosylation state, biological activity and stability and will be a matter of choice for the skilled addressee.

In addition to promoting expression of a NBEA polypeptide in cells, in certain circumstances it may be advantageous to substantially prevent or reduce the expression or activity of the native NBEA in a host, for example, for the production of animal models for use in drug screening, or particularly if the native NBEA is of a mutant form. Thus, according to a further aspect of the invention, there is provided an antisense nucleotide fragment complementary to a NBEA nucleotide sequence of the present invention. Included in the scope of ‘antisense nucleotide fragment” is the use of synthetic oligonucleotide sequences or of equivalent chemical entities known to those skilled in the art, for example, peptide nucleic acids.

Further, such sequences can be used as part of ribozyme and/ortriple helix sequences, which may also be useful for target gene regulation. Also provided is a nucleotide fragment comprising a nucleotide sequence which, when transcribed by the cell, produces such an antisense fragment, Typically, antisense RNA fragments will be provided which bind to complementary NBEA mRNA fragments to form RNA double helices, allowing RNAse H to cleave the molecule and rendering it incapable of being translated by the cell into polypeptides.

A further aspect of the present invention provides ligands, preferably antibodies specific to the NBEA or epitopes thereof. Production and purification of antibodies specific to an antigen is a matter of ordinary skill, and the methods to be used are clear to those skilled in the art.

The term antibodies can include, but is not limited to, polyclonal antibodies, monoclonal antibodies (mAbs), humanised or chimeric antibodies, single chain antibodies, Fab fragments, F(ab′)2 fragments, fragments produced by a Fab expression library, anti-idiotypic (anti-id) antibodies, and epitope binding fragments of any of the above.

Such antibodies may be used in modulating the expression or activity of the full length or truncated NBEA polypeptide, or in detecting said polypeptide in vivo or in vitro.

The present invention further provides a recombinant or synthetic NBEA polypeptide for the manufacture of reagents for use as prophylactic or therapeutic agents in mammals. In particular, the invention provides pharmaceutical compositions comprising the recombinant or synthetic NBEA polypeptide together with a pharmaceutically acceptable carrier therefor.

There is also provided use of a polypeptide or nucleic acid sequence as herein before described in preventing, delaying, treating or inhibiting neural system disorder such as autism. There is further provided a method of preventing, delaying, treating or inhibiting neural system disorders comprising providing NBEA to a subject displaying or predicted to display neural system disorders such as autism.

A yet further aspect of the present invention provides use of polypeptides or nucleic acid sequences as herein before described in the treatment of neuronal system disorders system, such as austim. One such envisaged treatment may be by way of so-called gene therapy in which a wild-type NBEA gene, or a or an allelic variant, minigene, a synthetic gene or a homologue thereof is introduced to a subject possessing a mutant NBEA gene in order to counter the effects of the mutant NBEA gene. This may be performed by the implantation of cells, such as fibroblasts expressing human or mammalian NBEA fused to herpes virus VP22 protein that will transfer itself and NBEA into adjacent neurons. Transformation of the cells to be implanted may be performed in vitro by any number of techniques, including physical means such as microinjection, electroporation, bioballistic or particle bombardment, jet injection or others; by chemical means such as using calcium phosphate, DEAE dextran, polylysine conjugates, ‘starburst” dendrimer conjugates, polybrene-dimethyl sulphoxide.

The NBEA gene itself or a or an allelic variant, minigene, a synthetic gene or a homologue, within an appropriate vector end-linked to an appropriate expression system, may be directly delivered via receptor-mediated uptake systems such as asialoglycoprotein and transferrin, liposomes, virus-like particles, intracellular targeting ligands and others; and by biological means including retroviral vectors such as Moloney murine leukaemia virus, adenovirus vectors and adeno-associated virus vectors, Herpes Simplex virus vectors, Semliki Forest virus vectors, Sindbis virus vectors, lentivirus vectors and others.

Furthermore, the invention provides methods for evaluating the efficacy of drugs for such disorders and monitoring the progress of patients involved in clinical trials for the treatment of such disorders.

The invention further provides methods for the identification of agents which modulate the expression of a mutated or wild-type NBEA gene and/or the activity of the product(s) of such a mutant or wild-type NBEA gene which is involved in processes relevant to neural system disorder such as autism. Such agents may include agonists, defined as agents which increase the expression of a mutated or wild-type NBEA gene and/or activity of the product(s) of such a mutant or wild-type NBEA gene, and/or antagonists, defined as agents which decrease the expression of a mutated or wild-type NBEA gene and/or the activity of the product(s) of such a mutant or wild-type NBEA gene, Thus, the present invention in a further aspect also provides agonists and/or antagonists.

The biological function of the NBEA gene can be more directly assessed by utilizing relevant in vivo and in vitro systems, in vivo systems can include, but are not limited to, animal systems which naturally exhibit the symptoms of nervous system disorders, or ones which have been engineered to exhibit such symptoms.

Further, such systems can include, but are not limited to; transgenic animal systems, in vitro systems can include, but are not limited to, cell-based systems comprising NBEA gene/NBEA protein expressing cell types. The cells can be wild type cells, or can be non-wild type cells containing modifications known or suspected of contributing to the neuronal system disorder of interest.

In further characterising the biological function of the NBEA mutant or wild-type gene, the expression of the NBEA mutant or wild-type gene can be modulated within the in vivo and/or in vitro systems, i.e. either overexpressed or underexpressed in, for example, transgenic animals and/or cell lines, and its subsequent effect on the system can then be assayed.

Alternatively, the activity of the product of the identified gene can be modulated by either increasing or decreasing the level of activity in the in vivo and/or in vitro system of interest, and its subsequent effect then assayed. The information obtained through such characterisations can suggest relevant methods for the treatment or control of nervous system disorders. For example, relevant treatment can include a modulation of gene expression and/or gene product activity.

Characterisation procedures such as those described herein can indicate whether such modulation should be positive or negative. As used herein, ‘positive modulation” refers to an increase in gene expression or activity of the gene or gene product of interest. ‘Negative modulation’, as used herein, refers to a decrease in gene expression or activity.

In vitro systems can be designed to identify agents capable of binding the NBEA mutant or wild-type gene products of the invention. Agents identified, for example, could be useful in modulating the activity of wild type or mutant NBEA gene products, could be useful in elaborating the biological function of the NBEA gene products, or could disrupt or enhance normal NBEA gene product interactions, for example, the activators or inhibitors of NBEA protein as disclosed in Keenan etal, 1997, FEBS Letters, A15-pp 101-108. Such agents may be investigated for their use in treating or alleviating autism

The present invention is explained in greater detail in the following non-limiting examples.

### EXAMPLE 1

Disruption of the NBEA-Gene in a Patient with Autism

A detailed molecular genetic analysis of an autistic male, with a de novo balanced translocation t(5;13)(q13.3;q14.3) was completed. The patient has no severe mental retardation and none of his relatives exhibit symptoms of autism.

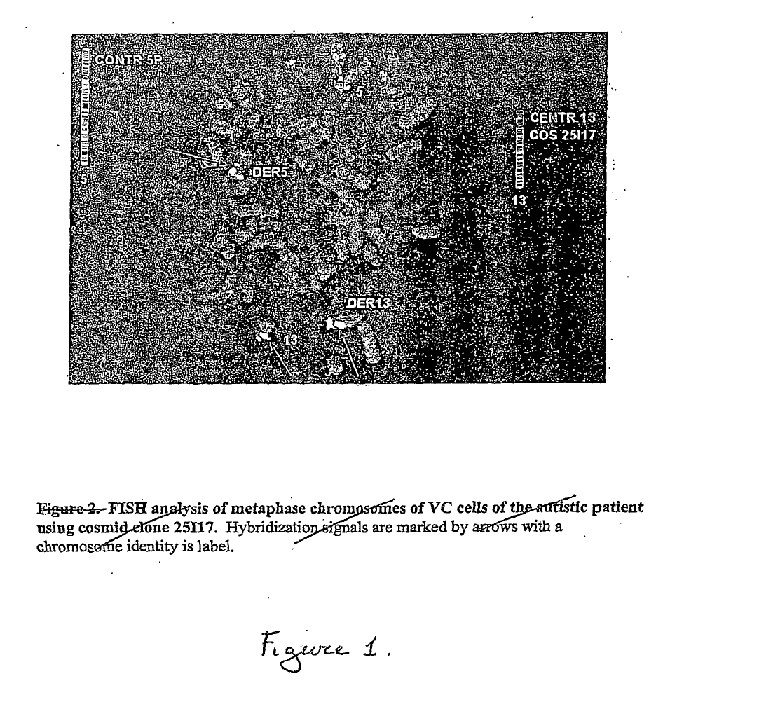

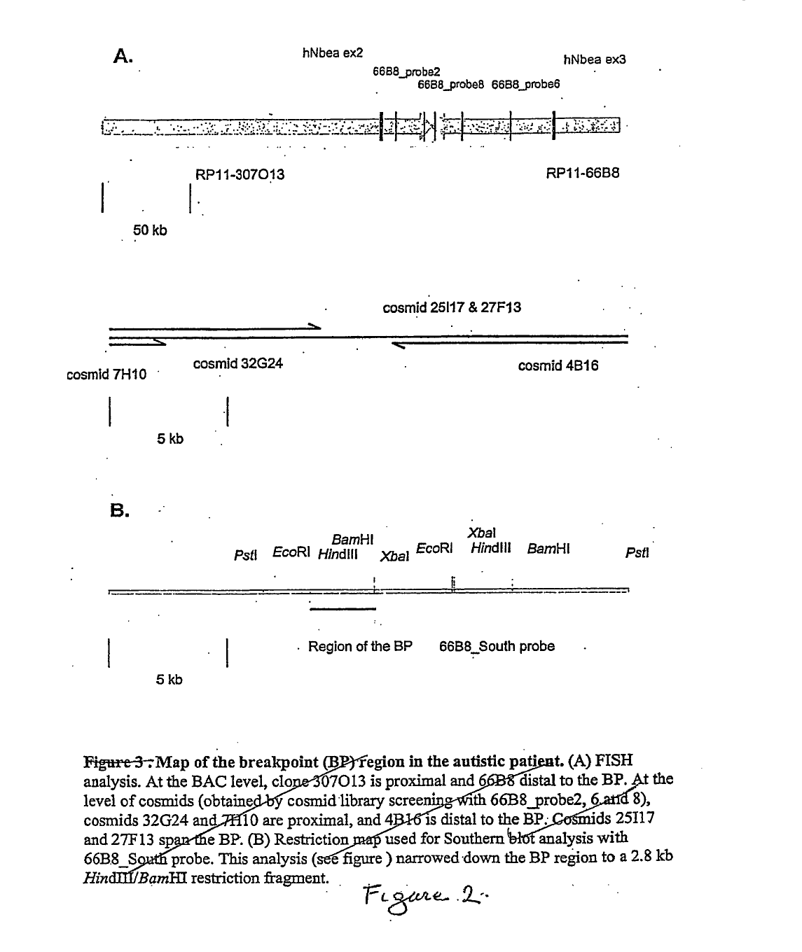

FISH analysis, by means of BACs hybridized against prometaphase chromosomes of the patient, was first used to delineate the rearrangement sites involved in the translocation event: clones RP11-307O13 (AL138690) and RP11-66B8 (AL161902) are proximal and distal to the breakpoint, respectively (data not shown). These clones overlap for 100 bp only and are part of the 10523-kb contig NT_{—}009984, for which the complete DNA sequence is known (GenBank accession number GI: 22052081). Cosmids corresponding to probes located within these BACs were isolated by cosmid library screening and used for FISH analysis. 25I17 cosmid, fished with 66B8-probe8 (primers: 66B8_probe8S, 5′-CTGCCTGCTTCCCTGGATTCAG-3′, 66B8_probe8AS, 5′-ATGGTGCATGGCTCTCACAGAG AG-3′), was shown to span the breakpoint on chromosome 13 (FIG. 1). The extremities of the cosmid clone were sequenced in order to map it on the breakpoint region (FIG. 2).

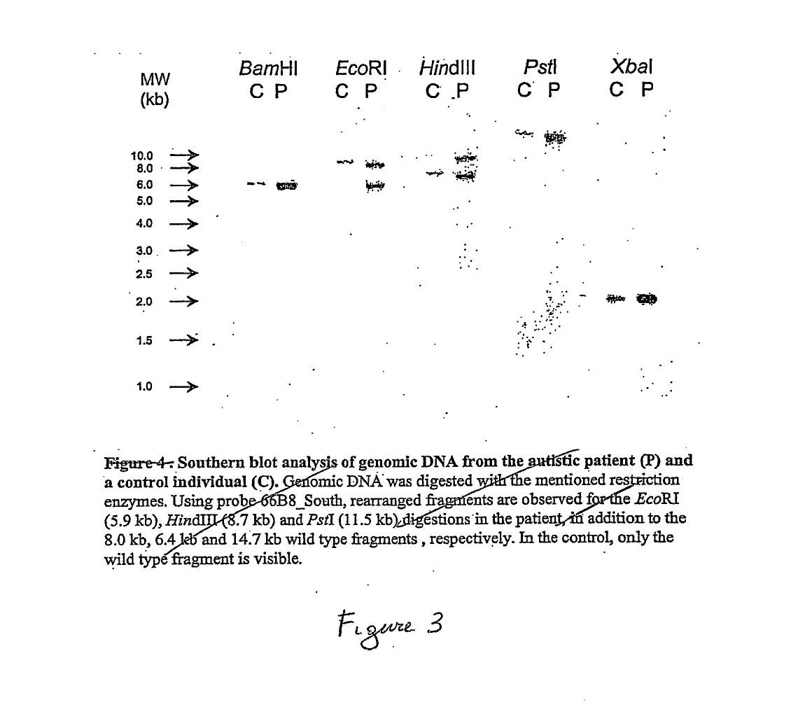

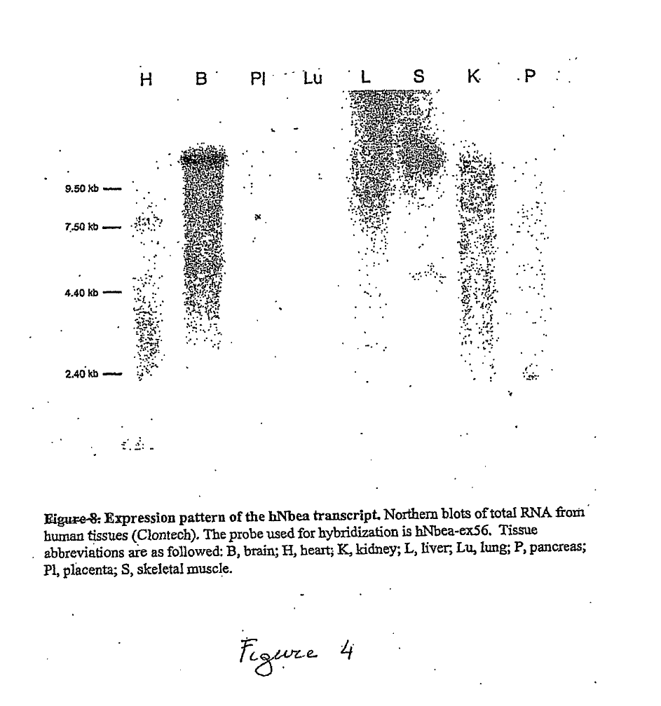

Southern blot hybridization analysis with DNA from the patient and a normal control digested with various restriction enzymes revealed a 5.9 kb EcoRI, a 8.7 kb HindIII and a 11.5 kb PstI additional rearrangement fragments in the patient's genomic DNA (FIG. 3) when using 66B8-South probe (primers: 66B8_South probe_S, 5′-TCCATTTGTTTCATCACCACTTGTGG-3′, 66B8_South probe_AS, 5′-CATGTMCMGTCAATCTCCTCTTCCCC-3′), indicating that the breakpoint is located within a 2.8 kb HindIII/BamHI restriction fragment (FIG. 2).

Database search analysis using BLAST (National Center for Biotechnology Information) revealed that neurobeachin is located in the vicinity of the chromosome 13q12 breakpoint. A blastn of the complete mouse cDNA neurobeachin sequence (10949 nt) against the human genome revealed homology along contig NT_{—}009984, corresponding to 58 exons of the human orthologue of neurobeachin, spanning 730 kb of the human genome; it is disrupted by the breakpoint on 13q12, in intron 2. Recently, the full cDNA sequence of the human NBEA(NM 015678), also called BCL8B (AF467288) has been published in Genbank_(GenBank accession number GI: 21536251 and 21434742, respectively).

### EXAMPLE 2

Isolation of a Full Length NBEA cDNA

The full-length cDNA of the NBEA gene was cloned by RT-PCR. The sequence was assembled in a consensus transcript of 10812 nt in length, in full agreement with the published sequences. This sequence predicted a continuous open reading frame (ORF) of 8838 nt translated in a protein of 2946 aa. Moreover, a CpG island encompassing. 1456 bp of NBEA was identified (http://l25.itba. mi.cnr.it/genebin/wwwcpg.pl). A putative promoter was also found within this region using the Promoter Inspector program (http://genomatix.gsf.de/cgi-bin/).

### EXAMPLE 3

Preparation of Rabbit Antisera Reactive with NBEA Proteins from Mouse and Human

Standard protocol of rabbit immunization with peptides was used by Eurogentec to produce 3 antisera reactive with the human and the mouse native NBEA. The pure peptides were coupled to the Keyhole Limpet Hemocyanin carrier protein. The antisera are immunoreactive with NBEA epitopes not present on other human proteins. The antisera can immunoprecipitate NBEA proteins from solution as well as react with NBEA protein on Western blots of polyacrylamide gels. The antisera also react with the native endogenous NBEA when used for immunocytochemistry in cell-lines or in primary cultures of neurons as well as for immunohistochemistry on mouse sections. The sequences of the peptides used for the production of the 3 anti-NBEA antisera are:

| | |
|---|---|
| anti-NBEA1: | KVSDDILGNSDRPGS |
| anti-NBEA2: | IEDLSQSQSPESETDY |
| anti-NBEA3: | YPGCDAGIRAMDLSHD |

### EXAMPLE 4

Brain Specific Expression of NBEA

Expression analysis, by mean of Northern blot revealed an abundant transcript of 11.9 kb in the human brain (FIG. 4), which is in agreement with the brain-specific expression pattern of the mouse Nbea (Wang et al., J. Neurosci., vol. 20, p. 8551, 2000) and with the expected length of the transcript. NBEA is also slightly expressed in the skeletal muscles, the kidney and the heart. No expression could be detected in the placenta, the lung, the liver or the pancreas.

Expression of neurobeachin in the adult mouse and during mouse development was further investigated by in situ hybridization (whole mount and on sections) and immunohistochemistry analyses. For in situ hybridization experiments two different riboprobes, choosen along the cDNA sequence of NBEA, were used. Rabbit polyclonal antibodies directed against two different epitopes of NBEA were applied in immunohistochemistry experiments.

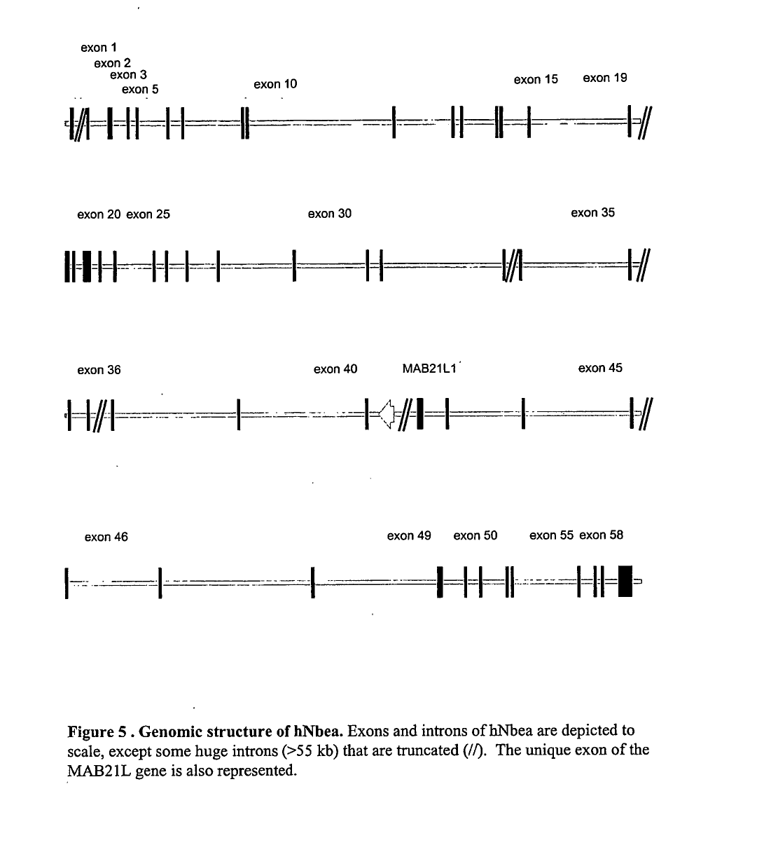

Both techniques gave an overlapping pattern of expression, assuring specificity. Whole mount in situ hybridizations of developing embryos showed that NBEA is expressed at stages as early as E7.5 (also detected by RT-PCR). In these early stages of development (E7.5-E9.5) the expression of NBEA is rather ubiquitous (FIG. 5a). Later stages (E10.5-E12.5) revealed a more specific pattern of expression in the branchial arches, the nose, the limbs and the tail (FIG. 5b).

However on slides of the late developing embryo (E15-E17) the expression appeared to be rather limited the cells of the CNS and derivative tissues and especially in cells of the cortical plate that have already migrated and are differentiated into neurons (data not shown):

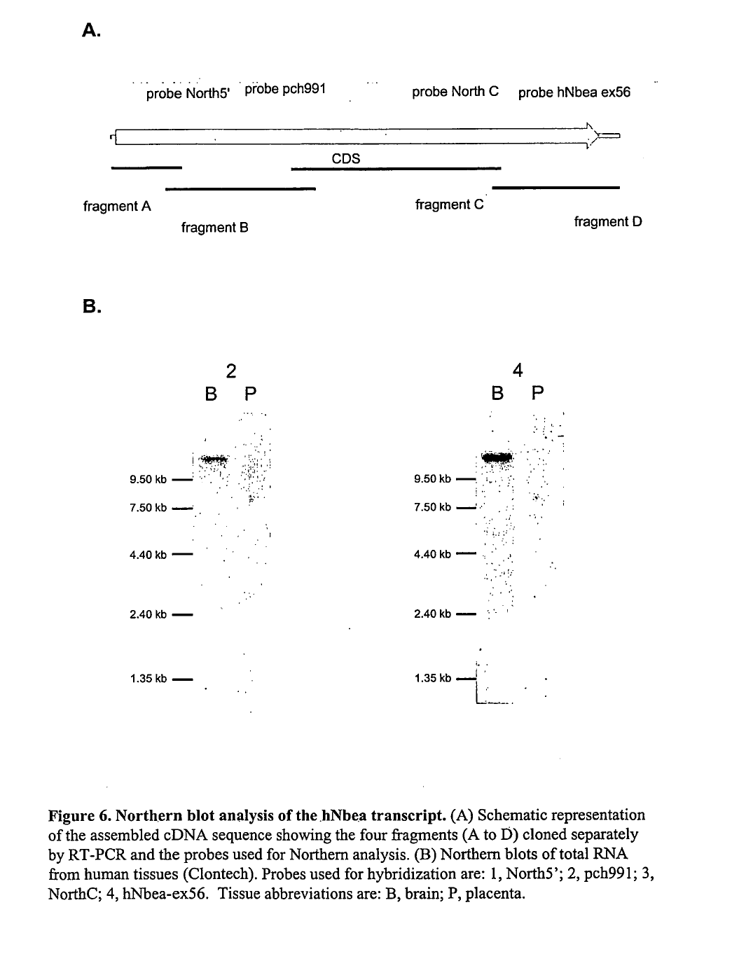

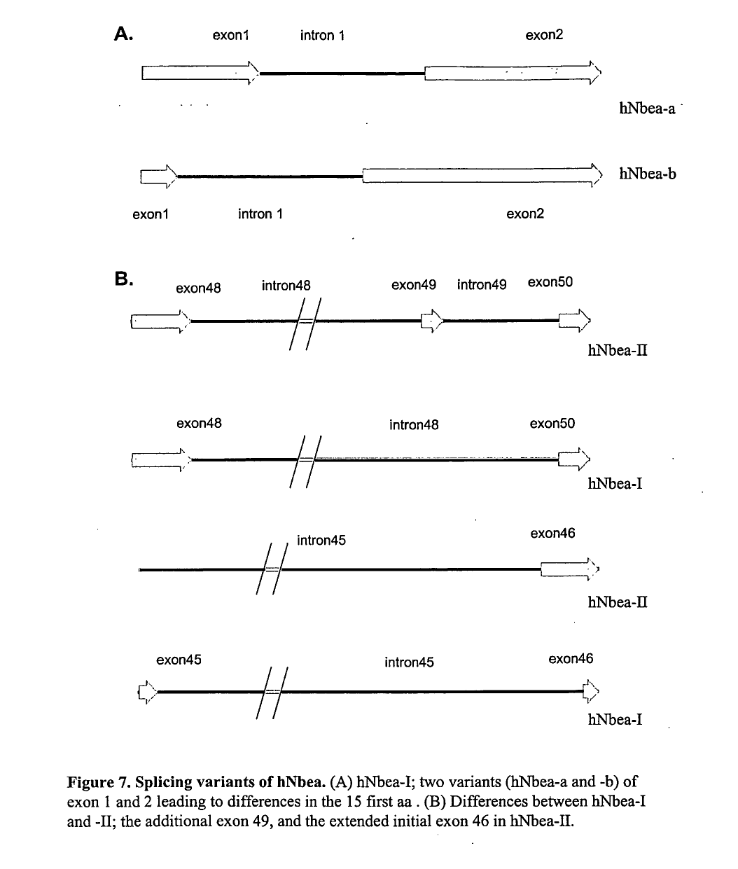

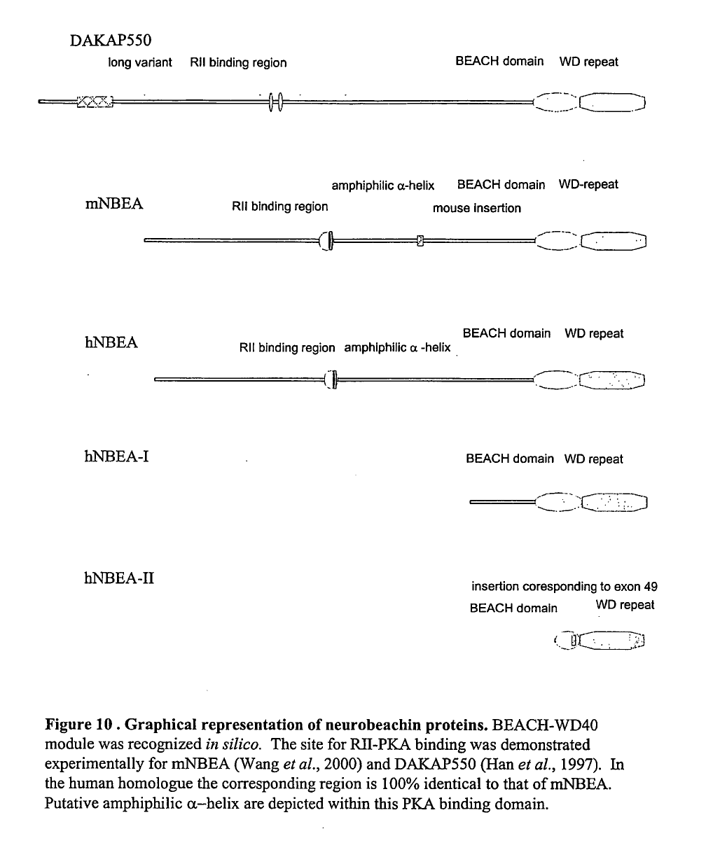

In adult brain neurobeachin is broadly expressed, but very specific in neuronal cells. Particularly high expression was observed In the hippocampus (FIG. 6), in the cerebellum (FIG. 7) and in the cerebral cortex.

### EXAMPLE 5

Identification of MARCKS and PKA as Interacting Partners of Neurobeachin by Yeast Two-Hybrid Screening

The Matchmaker Two-hybrid System 2 was purchased from CLONTECH (Palo Alto, Calif.). All experiments were performed in the yeast reporter strain CG-1 945 (Trp⁻ and Leu⁻). The “bait” constructs consisted of parts of the human. NBEA cloned into the yeast vector pGBKT7 (CLONTECH). Bait pvw4b comprises the PKA binding domain of NBEA, i.e. amino acids 1029 to 1473, bait pvw8b comprises the BEACH domain and the WD-40 repeats, i.e. amino acids 2254 to 2946, bait pvw9 comprises the PH-like and the BEACH domains, i.e. amino acids 2129 to 2614, bait pvw9b comprises the PH-like and the BEACH domains and the WD-40 repeats, i.e. amino acids 2129 to 2946. The pGBKT7 vector allows the fusion of the protein of interest to the C-terminal end of the GAL4 DNA-binding domain and contains TRP1^{and} Kan^{r} reporter genes for selection of transformants. The NBEA bait constructs did not show autonomous transcriptional activation and hence were good candidates for the detection of protein interactions in the yeast two-hybrid transcriptional activation assay. An oligo(dT)- and randomly primed “prey” cDNA library from 12.5-day-old embryonic mice cloned into the pACT2 vector were obtained from the University of Leuven and Flanders Interuniversity Institute for Biotechnology, Belgium. The pACT2 vector allows the fusion of proteins to the C-terminal end of the major GAL4 activation domain and contains LEU2 and Amp^{r} for selection of transformants. 1×10⁹ CG-1945 yeast were transformed with 66 μg of bait-DNA and 33 μg of prey-library-DNA using a LiAc high efficiency transformation protocol (Gietz, R. D. Yeast 1995; 11: 355-360). This yeast strain contains the HIS3 and lacZ reporter genes under the control of promoters containing GAL4 binding sites. Transformants were grown for 10 days at 30° C. on triple selective (lacking Trp, Leu, and His) synthetic dropout (SD⁻) agar plates containing 10 mM 3-aminotriazol (Sigma). Double transformed His⁺ yeast colonies were restreaked on new SD⁻agar plates and grown for another 24-48 h. For the qualitative measurement of β-galactosidase activity, colony lift filter assays were performed according to standard protocols. Plasmid DNA was isolated from positive (blue) colonies by glass bead lysis, extraction with phenol/chloroform, and ethanol precipitation and subsequently used to transform the Escherichia coli by electroporation. pACT2 plasmids containing different inserts as analyzed by PCR amplification and BglII digestion were reassayed by cotransformation into yeast-competent cells with either the bait construct used to fish the candidate, or an irrelevant bait construct, or the empty pGBKT7 vector. Plasmids that generated colonies on SD⁻ agar plates and were positive in the X-gal filter assay with the NBEA bait constructs were considered for further analysis. In this way, the mouse myristoylated alanine-rich C kinase substrate (Marcks, fished with bait pvw8b and pvw9b) and protein kinase A (PKA, fished with bait pvw6c) were identified as most probable interacting partners of NBEA. The interaction with PKA is in agreement with the surface plasmon resonance measurements experiments of co-immunoprecipitations of Wang et al. (J. Neurosci., vol. 20, p. 8551, 2000).

### EXAMPLE 6

Yeast Expression of Neurobeachin

Yeast deletion strains: Genomic deletions of specific genes can be made in the S. cerevisiae W303-1A strain (Thomas, B. J. and Rothstein, R. J., Cell 1989; 56: 619-630), the BY4741 strain (Brachmann et al., Yeast 1998; 14: 115-132) or the □1278b strain. (Kron, S. J. Trends Microbiol. 1997; 5:450-454) as indicated. They are obtainable by PCR product-directed gene disruption as described previously (Brachmann et al., Yeast 1998; 14: 115-132) using oligonucleotides described in WO02068663A1, and using the pRS vectors as templates for auxotrophic selectable markers. Deletions are checked by Southern Blot analysis (Sambrook et al. Molecular Cloning, a Laboratory Manual, 2nd edn. 1989; Cold Spring Harbor, N.Y.: Cold Spring Harbor Laboratory Press) or by PCR analysis. The PKA deletion strains ASY62 and ASY63 were kindly provided by S. Garrett and have been described previously (Smith A. et al., EMBO J., 1998; 17:3556-3564). The NBEA cDNA is transformed in yeast as recombinant constructs that contained the triose phosphate isomerase (TPI) promoter (Alber T, Kawasaki G., J Mol Appl Genet 1982; 1:419-434), allowing the NBEA-cDNAs to be constitutively expressed. For this purpose, the n eurobeachin cDNA, can be ligated into the EcoRI-Xho1 sites of the yeast/E. coli shuttle vector pJW212 which is a derivative of pYX212 (R&D systems Europe Ltd., Abingdon, UK). The cloning of the cDNA inserts is confirmed by sequence analysis using a method based on the standard dideoxy sequence analysis (Sanger et al., Proc. Natl. Acad. Sci. USA 1977; 74: 5463-5467). The resulting neurobeachin-expression plasmids is transformed into the appropriate yeast strains according to the protocol outlined by Gietz R. D. and Schiestl R. H. (Methods in Molecular and Cellular Biology 1995; 5: 255-269). Transformed cells are plated on selective glucose-containing medium without uracil (SD-ura) as specified by Sherman et al. (Methods in Yeast Genetics. 1986; Cold Spring Harbor, N.Y.: Cold Spring Harbor Laboratory Press).

Yeast culture: Yeast cells are cultured in YEP medium (2% (w/v) bacto peptone, 2% (w/v) yeast extract) or in the appropriate selective medium in order to maintain plasmids in transformed strains. Media are supplemented with 4% (w/v) glucose (YPD or SD). For pseudohyphal growth, cells are plated on nitrogen limitation medium (1 mM asparagine, 0.17% (w/v) yeast nitrogen base without amino acids and without NH₄SO₄, 2% (w/v) glucose and 1.5% (w/v) agar). Cells are mostly grown at 30° C. or 25° C. for different time periods as specified.

Preparation of crude extracts for Western bloting: Yeast cells are inoculated at a density of OD₆₀₀ of 0.2 in 5 ml selective medium and grown for 16 hours at 30° C. One milliliter of the culture is then transferred to a microcentrifuge tube and chilled on ice. After fast harvesting of the cells by centrifugation in a cooled (4° C.) microcentrifuge at maximal speed for 15 s and the pellet are resuspended in 50 μl prewarmed (95° C.) standard SDS-PAGE sample buffer. After boiling the mixture for 15 min in order to denature and inactivate all enzymes it then are processed by Western blot analysis as described below.

Western Blotting: The denatured and reduced protein mixtures are separated by SDS-PAGE as performed under reducing conditions on either 4-20% linear gradient gels or on 8% or 12% homogenous gels (Novex, San Diego, Calif.). After electrophoresis, the proteins are normally electrophoretically transferred to nitro-cellulose filters (Hybond-C, Amersham, UK) or to PVDF filters (ABI, San Fransisco, Calif.). The filters are blocked by incubation for 1 hour in PBS with 0.05% (v/v)Tween 20 and 5% (w/v) skimmed dried milk (blocking buffer). The filters are then incubated overnight with a specified monoclonal antibody or a specified polyclonal antiserum appropriately diluted in same blocking buffer. After washing the filters three times in Tween-PBS and they can be treated for 1.5 h at room temperature with horseradish peroxidase-labelled rabbit anti-mouse IgG (Dakopatts, Denmark) diluted 1/3000 in blocking buffer. After three washes in Tween-PBS, streptavidine-biotinylated horseradish peroxidase complex (Amersham), diluted 1/250 in blocking buffer, are applied for 1.5 h at room temperature. Thereafter, the filters are usually washed three times in Tween-PBS and once in PBS. And the filters are then to be incubated in PBS containing 0.05% (w/v) diaminobenzidine and 0.03% (v/v) hydrogen peroxide until background staining develops.

It should be clear that the formation of an immunological complex between the monoclonal antibodies and the antigen is not limited to the precise conditions described above, but that all techniques that respect the immunochemical properties of the antibody and antigen binding will produce similar formation of an immunological complex.

The detection of the immunologically bound monoclonal antibody can be achieved by conventional technology known and comprised in the art, with a second antibody that itself carries a marker or a chemical or physical group as a marker.

Microscopy: Yeast cells grown on pseudohyphae-inducing nitrogen limitation medium are scraped from plate, mixed with water and mounted on glass slides. Images can be processed with a laser microscope (e.g. ZEISS-axioplan) under a 100× oil-immersion objective.

### EXAMPLE 7

Lentiviral Transfer and Locoregional Expression of Neurobeachin siRNA in the Brain

The present invention also involves in vivo post-transcriptional silencing (RNA interference (RNAi)) of the NBEA gene.

More particularly the present invention involves the use of a lentiviral vector-based system for expressing short hairpin RNAi to silence NBEA locoregionally in the brains of animal models To avoid the non-specific cellular responses to double-stranded RNA in mammalian cells, small interfering RNAs (siRNAs) or short-hairpin RNAs (shRNAs) are designed as described by Elbashir, S. M. et al. Duplexes of 21-nucleotide RNAs mediate RNA interference in cultured mammalian cells. Nature 411, 494-498 (2001)).

By lentiviral vector-based system (Lentiviral vectors (LV)), such as those derived from the human immunodeficiency virus type 1 (HIV-1) which are currently used, stable gene transfer is mediated both in dividing and non-dividing cells (Naldini, L. et al. In vivo gene delivery and stable transduction of nondividing cells by a lentiviral vector. Science 272, 263-7 (1996) and Baekelandt, V. et al. Characterization of lentiviral vector-mediated gene transfer in adult mouse brain. Hum. Gene Ther. 13, 841-853 (2002)). These vectors are engineered to express small hairpin RNAs (shRNAs) or double stranded RNA, which get processed in vivo into siRNAs-like molecules capable of carrying out NBEA specific silencing. The dsRNA corresponding with the NBEA gene in the cell will promote the destruction of mRNA produced by NBEA, thereby preventing its expression (Synthesis and in vivo use of such siRNAs as been described by McCaffrey, A. P. et al. RNA interference in adult mice. Nature 418, 38-39 (2002). Such siRNAs seem to be capable of distinguishing between target mRNAs with a single base mismatch.

Stereotactic injection of LV encoding a short-hairpin RNA specific for NBEA mRNA in mouse brain is capable of inhibiting expression of NBEA. As the mouse or rat is well validated model for studying human disease such locoreginal NBEA silenced mice or such locoreginal NBEA silenced mice are a suitable in vivo diagnostic tool or animal model to study autism The method can be used for other non-human mammalians, adult or embryonic.

Preparation of lentiviral vectors for lentiviral-mediated RNA interference has been described by Abbas-Terki,-T; et al. Hum-Gene-Ther. Dec. 10, 2002; 13(18): 2197-201 other suitable vector systems are described in Baekelandt-V. et al. “Characterization of lentiviral vector-mediated gene transfer in adult mouse brain.”

Such small interfering RNA (siRNA) encoded by these locoregional introduced vector systems and capable to selectively bind to NBEA mRNA causing its degradation, are introduced in the target tissue, such tissues are preferably neural and pore particularly the hippocampus, cerebellum and the cerebral cortex.

The locoregional NBEA silenced animal models in comparison with wild type animal models are used to elaborate the effects of disturbance NBEA signal transduction, whereunder disturbed PKA achoring or disturbed compartmentalization of PKA on autism pathology. Moreover the locoregional NBEA silenced animal models or the NBEA silenced cell lines (e.g. yeast, AtT-20, Neuro-2A, PC12 Cell) are used to measure effects on the cellular pathways that are controlled by NBEA and in particular the type II protein kinase A (PKA) phosphoration pathway, intracellular vesicular transport and membrane dynamics controlled and the effect of compounds of pharmaceuticals thereon.

### EXAMPLE 8

### Cellulalr Assays

Secretion Studies

Transfection of cell with NBEA is for instance done by retroviral transduction of cells using pMSCV constructs. The 4 μg pMSCV construct is transfected together with 6 μg helper plasmid (plK Ecopac) using 30 μl of fugene in a 10 cm dish of HEK 293 at 60-70% confluency (include Mock transfection). 24 h later the medium is replaced with 3.5 ml fresh medium per dish. Again 24 h later 3.5 ml virus is taken of containing medium with a 10 ml syringe. A special low protein absorbance filter (0.45 μm) on needle is put and the virus containing medium is pushed trough filter and collected in 4 cryotubes (˜0.8 ml/tube), snap frozen tube using liquid nitrogen and if required. Stored at −80° C. in the right place (boxes marked for viral content).

For viral transduction of target cells (typically mouse ES or mouse embryonic fibroblasts, use cells of early passage number) medium is taken from cells in a 6-well plate at 70-80% confluency (seed different concentrations of cells, the most ideal is selected). 1 ml of fresh medium is put on cells (FMEM/F12+10% FBS for MEFs) and 2 μl polybrene (8 μg/μl) and ˜0.8 ml virus is added. The mixture is equally distributed over cells by gently shaking the dish. The cells are incubated at 37° C., 5% CO2. After 24 hours, the medium is refreshed containing selection antibodies (typically puromycine at 5 μg/ml). Two passages or more are carried in the in the virus lab. After 24 hours the cell death is checked in the non-transfected cells. When most of the non-transfected cells are dead, selection is stopped. Some of the stable cells are froozen down at early passages

Cells (8-10×105) are transfected with the NBEA (or a gen allelic variant, minigene or a homologue thereof, that encodes for NBEA) using standard procedures as described above and are incubated overnight in serum-free Dulbecco's modified Eagle's medium (optional: supplemented with 10 μM Rp-cAMP [Biolog, Bremen, Germany]), a protein kinase A inhibitor.

The cells are washed and incubated for 1-3 h in fresh serum free medium (unstimulated fraction) and subsequently incubated for the same time in medium the presence of the secretagogues 10 μM forskolin (Sigma) and 0.1 mM IBMX (Sigma) or the carrier solvent (0.01% dimethyl sulfoxide). Alternatively, 60 mM KCl is used as secretagogue. The medium contains the stimulated fraction. The lysate of the cells can be used to normalize. This protocol was developed to minimize the tonic release of stored material induced by serum factors while maximizing the response to elevations in intracellular cyclic AMP (or direct membrane depolarization by KCl). The released proteins are concentrated by precipitated (e.g. by ice-cold 5%_(w/v) trichloroacetic acid or 4 volumes of −20° C. methanol) and then solubilized in SDS-PAGE sample buffer. Cells are lysed directly in sample buffer and run in parallel on 7.5% SDS-PAGE gels. The electrophoresed proteins are transferred to nitrocellulose and probed with a marker protein for dense-core secretroy granules like carboxypeptidase E or PC1 followed by a peroxidase-conjugated second antibody and chemoluminescence detection.

RNAi experiments are performed to reduce functional expression of NBEA (or a gen allelic variant, minigene or a homologue thereof, that encodes for NBEA) in cells with a regulated secretory pathway, like neuronal and endocrine cells. Subsequently, regluated secretion is analyzed using the above-described assay.

The haploinsufficiency of these NBEA interfered with normal secretion of dense-core secretory granules in neurons. This can be at various levels like granule biogenesis or maturation (e.g. acidification), transport, priming and membrane fusion, but also at the level of impaired signal tranduction after stimulation with an extracellular secretagogue. Complete ablation of NBEA results in a more severe phenotype like apoptosis while the correct titration of NBEA expression covers a milder phenotype.

Neurite Outgrowth Assay:

Neuritogenesis is important for many aspects of nervous system function and impaired outgrowth is therefore likely to cause pathology. The role of NBEA for autism is therefore tested for its role in neurite outgrowth. Primary cell cultures and/or cell lines are transduced with RNAi to lower expression of the candidate gene. Neurite outgrowth can be quantified e.g. by commerically avialable assay (e.g. Chemicon International).

Disruption of NBEA using small interfering RNA molecules specific to NBEA mRNA.

RNA interference with synthetic short RNA duplexes has been used to knock-down expression of endogenous genes in mammalian cells (Elbashir et al., 2002). We used short interfering RNA (siRNA) duplexes to study the effect of depletion of NBEA in NBEA transfected neuronal cells on the secretion of the dense-core secretory granules.

i): siRNA Preparation

All oligonucleotides for siRNA preparation are chemically synthetized by Dharmacon (CO, USA) and Xeragon (MD, USA) and were composed of ribonucleotides (A,U,G and C) plus a pair of 3′-terminal desoxy-thymidines (dTdT).

Small interfering RNA molecules (siRNA) are prepared by annealing pairs of oligonucleotides to design siRNA molecules that are specific for NBEA mRNA and contain an anti-sense strand that can hybridize to the target mRNA.

ii. Transfection of the NBEA Expressing Cells with the siRNA Molecules

The NBEA expressing cells are seeded into 8 well Nunc LabTekII chamber glass slides (VWR International) and aretransfected at a confluency of about 25% with each siRNA using GeneSilencer, a liposomal transfection reagent purchased from GTS (Gene Therapy Systems, CA, USA). Transfection is done according to the manufacturer's recommendations. Briefly, for transfection of each well, 0.3 μg of RNA and 1.75 μl of the GeneSilencer reagent are used to transfect each well of the slide.

RNAi experiments are performed to reduce functional expression of NBEA in Neuro 2a, PC12 Cell and AtT-20 Cell to study the regulated secretory pathway, which analyzed using the above-described assay.

Cell Cultures

AtT-20 (mouse tumor cells of the pituitary, ATCC CRL-1795) or Neuro-2A (mouse neuroblastoma cells, ATCC CCL-131) or SK-N-SH cells (human neuroblastoma cells, ATCC HTB11) are maintained in Dulbecco's modified Eagle's medium (DMEM) with glutamax (Invitrogen, Belgium) containing 10% foetal calf serum (FCS), 0.12% (v/w) sodium carbonate (Invitrogen) and 20 μg/ml gentamycin (Invitrogen) in a 5% humidified CO₂ atmosphere.

PC12 Cell (American Tissue Type Cell Collection (Rockville, Mass., USA)) are cultures according to L. A. Greene, J. M. Aletta, A. Rukenstein and S. H. Green (1987) PC12 pheochromocytoma cells: culture, nerve growth factor treatment, and experimental exploitation. Methods Enzymol. 147:207-216.

### EXAMPLE 8

### Primers Used for Deletion Analysis of the NBEA Gene

Multiplex Primers NBEA (Tann. ˜57.2-58)

| | | |
|---|---|---|
| NbeaMB- | ctggttggtggagaatttgacttgg | 189 bp |
| 1S | | |
| NbeaMB- | ttcaattagcccaacttctgtgctagtc | |
| 1AS | | |
| NbeaMB- | tgaagatcttctagttgatatgttggggg | 111 bp |
| 2S | | |
| NbeaMB- | cttacccagattccactttctcctcg | |
| 2AS | | |
| NbeaMB- | tcttttattgtttccccagccaagac | 125 bp |
| 3S | | |
| NbeaMB- | taaaacttaccgcagcgctacaacc | |
| 3AS | | |
| NbeaMM- | aagttattaacaatatcaacatgatccttaaaccag | 214 bp |
| 1S | | |
| NbeaMM- | gcccagtggaataaaatgtacactgactag | |
| 1AS | | |
| NbeaMM- | cccattactgggtatatacccaaaggac | 225 bp |
| 2S | | |
| NbeaMM- | tcatccatgtccctacaaaggacatg | |
| 2AS | | |
| NbeaMM- | tcacagtgtgcccaatatgctgc | 169 bp |
| 3S | | |
| NbeaMM- | acctatgagaaactgctccccaagc | |
| 3AS | | |
| NbeaME- | tttaatagccaattggtgctttgaacc | 139 bp |
| 1S | | |
| NbeaME- | agccaggataaagtagatgttgctgttg | |
| 1AS | | |
| NbeaME- | ttaggaacctttcacaaccttcttcctc | 100 bp |
| 2S | | |
| NbeaME- | actagttctccaagaccttgcaacgg | |
| 2AS | | |
| NbeaME- | ggaactaattccagagttctactacctaccagag | 154 bp |
| 3S | | |
| NbeaME- | catcctgttgatccgcacaaagtc | |
| 3AS | | |
| ContrM- | gcctagccaacatggtgaaacctc | 300 bp |
| 1S | | |
| ContrM- | caagggaggacagaaacagcaacag | |
| 1AS | | |

1-62. (canceled)

63. A method of testing or screening a mammal thought to have or to be predisposed to have a neural system disorder comprising detecting the presence of mutation in the NBEA gene or its associated promotor in a sample of said mammal.

64. The method of claim 63, said method comprising: detecting a modification of the NBEA gene or its promotor in the chromosomal material of said sample, wherein said modification is selected from a) substitution, b) deletion, c) frame-shift, d) insertion or e) altered epigenetic control; whereby said modification causes a loss of biological function in the NBEA gene product;

65. The method of claim 64, which further comprises, correlating the mutation of said gene with a potential for a neural system disorder.

66. The method of claim 63, wherein said mutation in the NBEA gene or its promotor is detected by hybridisation with a labelled probe.

67. The method of claim 63, wherein detection of the presence of the mutation in the NBEA gene is achieved by detecting altered levels of the mRNA transcripts or mRNA precursor.

68. The method of claim 63, wherein said mutation is detected by (a) amplification of the chromosomal material using PCR; (b) sequencing said material to detect the modification of the nucleotide sequence; and (c) correlating the modification of said gene with a potential for neural system disorders.

69. The method of claim 63, wherein, said method comprises: (a) detecting the absence, inappropriate, or modified expression of NBEA gene product using labelled ligands to said gene product in said sample; and (b) correlating said absence, inappropriate, or modified expression with a potential for neural system disorders.

70. The method of claim 69, wherein the said ligands are monoclonal or polyclonal antibodies.

71. The method of claim 63, wherein said neural system disorder is autism.

72. The method of claim 63, wherein said neural system disorder is selected from the group consisting of a disorder associated with any or several symptoms consisting of the group disturbed cognitive functions, disturbed emotional control, disturbed in motor control, a disorder resulting from a decreased number of Purkinje cells and a disorder resulting from brain anomalies.

73. The method of claim 63, wherein said neural system disorder results from a disturbed the glutamate neurotransmitter system.

74. The method of claim 63, wherein said neural system disorder results from reduced levels of the anti-apoptotic protein bcl2.

75. The method of claim 63, which comprises hybridising a polynucleotide sequence which is hybridisable with a variant NBEA gene, having a deletion, insertion or base substitution which affects transcription and/or translation of the NBEA gene to the NBEA gene in said sample.

76. A method of preventing or treating a neural system disorder in a mammal, said method comprising administering to said mammal a polynucleotide comprising the NBEA gene, an allelic variant, minigene or an homologue thereof encoding NBEA or an homologue thereof

77. The method of claim 76, wherein the neural system disorder is autism.

78. A method of preventing or treating a neural system disorder in a mammal, said method comprising administering to said mammal a polypeptide comprising NBEA or a fragment thereof.

79. The method of claim 78, wherein a neural system disorder is autism.

80. An isolated polynucleotide comprising a nucleotide sequence, wherein said sequence includes at least one mutation of the NBEA gene, wherein said mutation is selected from a) substitution, b) deletion, c) frame-shift, d) insertion, or e) site-directed mutagenesis that causes a loss of biological function in the NBEA gene.

81. An isolated cell containing the polynucleotide of claim 80.

82. The cell of claim 81, wherein said cell is a neural cell.

83. The neural cell of claim 82, wherein the cell is derived from an immortal cell line, such as embryonic stem cells, neuronal cell line, or tumour derived cell line.

84. The neural cell of claim 82, wherein the NBEA gene is under control of a neural-specific promoter or inducible promoters.

85. A non-human animal containing in its genome the polynucleotide of claim 80.

86. A vector containing the polynucleotide of claim 80.

87. An engineered cell comprising a vector comprising the vector of claim 86.

88. An engineered cell comprising a vector encoding RNAi specific for NBEA mRNA encoded by a heterologues gene relative to the genome of said cell.

89. A method of screening for therapeutic agents suitable to treat autism comprising: (A) providing the engineered cell of claim 87 or 88 or providing a cell containing an isolated polynucleotide comprising a nucleotide sequence, wherein said sequence includes at least one mutation of the NBEA gene, wherein said mutation is selected from a) substitution, b) deletion, c) frame-shift, d) insertion, or e) site-directed mutagenesis that causes a loss of biological function in the NBEA gene; (B) introducing to the cell a agent to be screened; and (C) correlating change in said cell with the activity of the agent.

90. The method of claim 89, wherein said changes in said cell are survival, proliferation, differentiation or outgrowth.

91. The method of claim 89, wherein said changes in said cell are changes the type II protein kinase A phosphorylation pathway.

92. A non-human animal with locoregional neural transgenes, wherein said animal comprises a vector encoding RNAi specific for NBEA mRNA encoded by a heterologues gene relative to the genome of said cell

93. A method of screening for therapeutic agents comprising: (A) providing the non-human animal of claim 85 or the non-human animal of claim 92 (B) introducing to the animal a agent to be screened; and (C) correlating a change in the development of autism.

94. A method of screening for a therapeutic agents suitable to treat autism comprising: (A) providing an engineered yeast cell, comprising an introduced nucleotide sequence comprising NBEA gene or an allelic variant, minigene, a synthetic gene or a homologue thereof; (B) introducing to the cell a compound, chemical signal or agent to be screened; and (C) correlating change in said cell with the activity of the compound, chemical signal or agent.

95. The method of claim 94, which method comprises screening for compounds, chemical signals or agents that directly or indirectly affect the biochemistry of NBEA.
